(19) United States
(12) Patent Application Publication
(10) Pub. No.: US 2022/0401301 A1
(43) Pub. Date: Dec. 22, 2022

(54) MEDICAL SYSTEM AND METHOD OF STERILITY TESTING THE MEDICAL SYSTEM

(71) Applicant: Roche Diabetes Care, Inc., Indianapolis, IN (US)

(72) Inventors: Hans List, Mannheim (DE); Stefan Riebel, Mannheim (DE); Andrea Rittinghaus, Neckarsteinach (DE)

(21) Appl. No.: 17/822,352

(22) Filed: Aug. 25, 2022

Related U.S. Application Data
(63) Continuation of application No. PCT/EP2021/054365, filed on Feb. 23, 2021.

(30) Foreign Application Priority Data
Feb. 26, 2020 (EP) 20 159 511.3

(51) Int. Cl. A61J 1/14; A61J 1/20

(52) U.S. Cl. CPC A61J 1/1406; A61J 1/2096; A61J 1/2006

(57) ABSTRACT

A medical system is disclosed. The medical system has a housing and a module received in the housing. The module includes an analyte sensor configured for detecting an analyte in a body fluid of a user, an electronics unit electrically connected to the analyte sensor, an insertion component configured for inserting the analyte sensor into body tissue of the user, and a sterility cap at least partially surrounding the insertion component. A removable protective cap is connected to the housing and covers the module. The protective cap at least partially surrounds the sterility cap. The sterility cap has a sterility testing access that has at least one of a septum and a multiple-step sealing. A method of sterility testing the medical system is also disclosed in which a rinsing liquid is inserted into an interior space of the sterility cap and microbial testing of the rinsing liquid is then completed.

### RELATED APPLICATIONS

This application is a continuation of PCT/EP2021/054365, filed Feb. 23, 2021, which claims priority to EP 20 159 511.3, filed Feb. 26, 2020, both of which are hereby incorporated herein by reference.

### BACKGROUND

This disclosure relates to a medical system and to methods of providing the medical system and of sterility testing of the medical system. The medical system may be used for detecting at least one analyte in a body fluid, such as a body fluid contained in a body tissue. The medical system may specifically be configured for the method of sterility testing. The medical system may be applied in the field of continuous monitoring of at least one analyte in a body fluid of a user, specifically applied in the field of home care and in the field of professional care, such as in hospitals. Other applications are feasible.

Monitoring certain body functions, more particularly monitoring one or more concentrations of at least one analyte concentration such as at least one metabolite concentration in a body fluid plays an important role in the prevention and treatment of various diseases. Such analytes can include by way of example, but not exclusively, glucose, lactate, cholesterol or other types of analytes and metabolites. Without restricting further possible applications, this disclosure will be described in the following text with reference to glucose monitoring. However, additionally or alternatively, this disclosure can also be applied to other types of analytes, such as the analytes mentioned above.

Generally, medical systems for long-term monitoring of an analyte in a body tissue of a user as well as corresponding insertion devices are known. As an example, WO 2019/122095 A1 discloses a medical system, comprising a housing and a preassembled functional module received in the housing. The preassembled functional module comprises an analytical sensor for detecting at least one analyte in a body fluid of a user, an electronics unit electrically connected to the analytical sensor and an insertion component for inserting the analytical sensor into a body tissue of the user. The medical system further comprises at least one removable protective cap connected to the housing, covering the preassembled functional module.

Despite the advantages achieved by the above-mentioned devices, several technical challenges remain. Specifically, medical products which may be delivered sterile to the customer may either be sterilized completely inside their packaging unit or the sterile compounds may be enclosed to the product kit in a separate sterile sealing. The user may have to assemble these devices prior to application.

In case no product kit shall be delivered to the customer but instead a fully integrated device comprising sterile parts and other parts which may be damaged by sterilization, the sterile parts may be further processed after the sterilization without any contamination. During the assembly of the device possible contamination might occur. Therefore, the complete product may have to be tested whether the sterile parts were contaminated or not, at least on a random basis. However, in these fully integrated devices, the sterile parts are generally no longer accessible. A removal of the sterile parts may require opening of the sterile sealing and, thus, may possibly cause contamination.

### SUMMARY

This disclosure teaches devices and methods which at least partially address the above-mentioned technical challenges. Specifically, a medical system is disclosed which allows for easy and user-friendly insertion of an analyte sensor into a body tissue and for continuous monitoring of an analyte in the body tissue of a user, while enabling the manufacturer to verify the sterility of sterile parts of these devices.

As used in the following, the terms “have,” “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B,” “A comprises B” and “A includes B” may both refer to a situation in which, besides B, no other element is present in A (i.e., a situation in which A solely and exclusively consists of B) and to a situation in which, besides B, one or more further elements are present in entity A, such as element C, elements C and D or even further elements.

Further, it shall be noted that the terms “at least one,” “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once. It shall also be understood for purposes of this disclosure and appended claims that, regardless of whether the phrases “one or more” or “at least one” precede an element or feature appearing in this disclosure or claims, such element or feature shall not receive a singular interpretation unless it is made explicit herein. By way of non-limiting example, the terms “sterility cap,” “protective cap,” “sterility testing access,” and “insertion component,” to name just a few, should be interpreted wherever they appear in this disclosure and claims to mean “at least one” or “one or more” regardless of whether they are introduced with the expressions “at least one” or “one or more.” All other terms used herein should be similarly interpreted unless it is made explicit that a singular interpretation is intended.

Further, as used in the following, the terms “preferably,” “more preferably,” “particularly,” “more particularly,” “specifically,” “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

In a first aspect of this disclosure, a medical system is disclosed. The medical system specifically may be configured for performing at least one medical function. Specifically and as will be outlined in further detail below, the medical system may comprise at least one part configured for qualitatively and/or quantitatively detecting at least one analyte in a body fluid of a user, such as one or more of glucose, triglycerides, lactate, cholesterol and/or other types of analytes. As will be further outlined in detail below, the medical system may further comprise one or more other components, such as protective components, components for insertion and the like.

The medical system comprises:
i) a housing;
ii) a preassembled functional module received in the housing, the preassembled functional module comprising
   a. an analyte sensor for detecting at least one analyte in a body fluid of a user;
   b. an electronics unit electrically connected to the analyte sensor; and
   c. an insertion component for inserting the analyte sensor into a body tissue of the user, specifically at least one of an insertion cannula and an insertion needle; and
iii) at least one removable protective cap connected to the housing, covering the preassembled functional module.

The preassembled functional module further comprises at least one sterility cap, which at least partially surrounds the insertion component, specifically the insertion component and at least a part of the analyte sensor. The at least one sterility cap is at least partially surrounded by the protective cap, specifically arranged at least partially within the protective cap. The sterility cap comprises at least one sterility testing access, the sterility testing access comprising at least one of a septum and a multiple-step sealing, specifically a two-step sealing.

The term “medical system” as used herein is a broad term and is to be given its ordinary and customary meaning to a person of ordinary skill in the art and is not to be limited to a special or customized meaning. The term specifically may refer, without limitation, to a system comprising at least one component configured for performing at least one medical function and/or for being used in at least one medical process, such as one or more of a therapeutic process, a diagnostic process or another medical process. Specifically, as outlined above, the medical system may be configured for and/or may comprise at least one component which is configured for being used in qualitatively and/or quantitatively detecting at least one analyte in a body fluid, such as in a body fluid contained in a body tissue of a user. The medical system specifically may be configured for performing at least one of the following two actions: the action of inserting an analyte sensor into the body tissue and the action of detecting the analyte in the body fluid by using the analyte sensor. The medical system may specifically be configured for continuously monitoring and/or detecting the analyte in the body fluid of the user. Additionally or alternatively, the medical system may comprise other components, such as components having a protective function and/or another medical function.

The term “detecting at least one analyte” as used herein is a broad term and is to be given its ordinary and customary meaning to a person of ordinary skill in the art and is not to be limited to a special or customized meaning. The term specifically may refer, without limitation, to a quantitative and/or qualitative determination of at least one analyte in an arbitrary sample or aliquot of a body fluid. For example, the body fluid may comprise one or more of blood, interstitial fluid or other types of body fluids. The at least one analyte, as an example, may be or may comprise one or more specific chemical compounds and/or other parameters, for example, one or more of glucose, triglycerides, lactate, cholesterol and/or other types of analytes. The result of the determination of the analyte may be or may comprise at least one numerical value indicating the presence and/or the concentration of the analyte in the body fluid, such as a single numerical value or, specifically in case of a monitoring, a sequence of numerical values, such as a sequence acquired over a period of time. For example, the result of the determination may be or may comprise at least one numerical indication of the analyte concentration in the body fluid, such as a glucose concentration.

Further, the medical system specifically may be, in a basic state before use, a unitary system which may be handled as one single piece. After use, which is after insertion of the analyte sensor into the body tissue, the medical system may disassemble into a disposable handling component including the insertion component in a used state as well as, optionally, at least one driving actuator as will be explained in further detail below, and into an analyte sensor unit, with a body mount, the analyte sensor and the electronics unit, wherein the body mount may be attached to the skin of the user and wherein the analyte sensor may protrude from the analyte sensor unit into the body tissue.

The term “housing” as used herein is a broad term and is to be given its ordinary and customary meaning to a person of ordinary skill in the art and is not to be limited to a special or customized meaning. The term specifically may refer, without limitation, to an arbitrary element which is configured for fully or partially enclosing one or more components and for providing protection for these one or more components, such as against mechanical influence and/or humidity. The housing, specifically, may be or may comprise a rigid housing, such as a rigid housing made of one or more of a plastic material, a metallic material or a cardboard material. The housing may have a base part which is configured for being disposed on the skin of the user, such as an essentially flat surface of the base part. The housing, as will be explained in further detail below, may comprise, may contain or may enclose fully or partially one or more further components, such as a driving actuator.

The term “functional module” (also referred to herein as simply “module”) as used herein is a broad term and is to be given its ordinary and customary meaning to a person of ordinary skill in the art and is not to be limited to a special or customized meaning. The term specifically may refer, without limitation, to a module, such as a unitary module made of one or more components, specifically a plurality of interconnected components, which are configured for interacting for performing at least one function and/or for being used for performing at least one function, specifically at least one medical function. In the present case, the functional module specifically may be a medical functional module configured for performing at least one medical function, such as for qualitatively and/or quantitatively detecting at least one analyte in a body fluid. For example, the functional module comprises the analyte sensor, the electronics unit and the insertion component, and, thus, may be configured for performing the at least one medical function of the medical system, such as detecting the at least one analyte in a body fluid. Thus, the functional module may comprise one or more components, such as the analyte sensor, which are inserted and remain in the body tissue of the user when the functional module performs the medical function of the medical system, such as detecting the at least one analyte in a body fluid. The functional module may also comprise one or more components, such as the insertion component, which are removed from the body tissue of the user after inserting the analyte sensor and, specifically, prior to performing the medical function of the medical system. The functional module is received in the housing as a preassembled functional module.

The term “preassembled” as used herein is a broad term and is to be given its ordinary and customary meaning to a person of ordinary skill in the art and is not to be limited to a special or customized meaning. The term specifically may refer, without limitation, to a state of a device having already completed an assembly process. Thus, as claimed, the medical system comprises the functional module as defined above in a preassembled state, which means the components of the functional module already being assembled, such as by being mechanically and/or electrically interconnected, thereby being ready for use for the at least one function, such as the at least one medical function, e.g., for the at least one analytical function. The preassembling specifically may take place in a factory, thereby rendering the preassembled functional module a factory-assembled functional module. Specifically, the medical system may be configured such that the at least one preassembled functional module is fully covered by the combination of the housing and the protective cap, such that the user may not see or manipulate the preassembled functional module without opening the medical device, e.g., without removing the protective cap.

The term “analyte sensor” as used herein is a broad term and is to be given its ordinary and customary meaning to a person of ordinary skill in the art and is not to be limited to a special or customized meaning. The term specifically may refer, without limitation, to an arbitrary element or device configured for detecting at least one condition and/or for measuring at least one measurement variable for analytical purposes. Specifically, the analyte sensor may be configured for qualitatively and/or quantitatively detecting at least one analyte in a body fluid of the user, such as one or more of the analytes listed above, more specifically glucose, in particular by interacting with the other components of the functional module, such as the electronics unit. The analyte sensor specifically may be configured for long-term and/or continuous monitoring of the analyte in a body fluid of a user. Thus, the analyte sensor may be configured for being at least partially implanted into a body tissue of a user. The analyte sensor, as an example, may be configured for being placed into the body tissue and for remaining therein for at least one week, by providing measurement data over this period of use. The analyte sensor specifically may be or may comprise an electrochemical analyte sensor.

The term “user” as used herein is a broad term and is to be given its ordinary and customary meaning to a person of ordinary skill in the art and is not to be limited to a special or customized meaning. The term specifically may refer, without limitation, to a person using the medical system. Specifically, the user may be a patient and/or a person intending to monitor the analyte, such as the glucose value, in one's own or another's body tissue. For example, the user may be a patient suffering from a disease, such as diabetes.

The term “electronics unit” (also referred to herein as “electronics”) as used herein is a broad term and is to be given its ordinary and customary meaning to a person of ordinary skill in the art and is not to be limited to a special or customized meaning. The term specifically may refer, without limitation, to a unit, such as a unit which may be handled as a single piece, which is configured for performing at least one electronic function. Specifically, the electronics unit may have at least one interface for being connected to the analyte sensor, wherein the electronics unit may provide at least one electronic function interacting with the analyte sensor, such as at least one measurement function. The electronics unit specifically may be configured for measuring at least one voltage and/or for measuring at least one current, thereby interacting with the analyte sensor, specifically the electrochemical analyte sensor. The electronics unit may further comprise at least one integrated circuit, such as a processor and/or a microcontroller. The term “processor,” as well as the term “microcontroller” as generally used herein are broad terms and are to be given their ordinary and customary meaning to a person of ordinary skill in the art and is not to be limited to a special or customized meaning. The terms specifically may refer, without limitation, to a device which is configured for performing calculations or logic operations. In particular, the processor may be configured for processing an electronic signal, such as a current or a voltage, specifically an electronic signal from the analyte sensor. Specifically, the processor and/or microcontroller may be or may comprise a central processing unit (CPU). Additionally or alternatively, the processor and/or microcontroller may be or may comprise a microprocessor, thus specifically the processor's elements may be contained in one single integrated circuitry (IC) chip. Additionally or alternatively, the processor may be or may comprise one or more application-specific integrated circuits (ASICs) and/or one or more field-programmable gate arrays (FPGAs) or the like. The processor and/or microcontroller specifically may be configured, such as by software programming, for performing one or more evaluation operations. Thus, the processor and/or microcontroller may be configured for processing and/or evaluating the electronic signal from the analyte sensor and, for example, outputting a signal indicating the analyte concentration measured by the analyte sensor. The electronics unit further may comprise at least one measuring device for measuring at least one of a voltage and a current, such as a potentiostat. The processor and/or the microcontroller specifically may be configured for controlling one or more electronic functions of the electronics unit.

The electronics unit specifically may comprise at least one electronics unit housing, wherein the analyte sensor, e.g., with a proximal end and/or an end providing electrical contacts for contacting the analyte sensor, may protrude into the electronics unit housing and may be electrically connected with at least one electronic component within the electronics unit housing. As an example, the proximal end and/or at least one contact portion of the analyte sensor may protrude into the electronics unit housing and, therein, may be electrically connected to at least one electronic component, such as to at least one printed circuit board and/or at least one contact portion of the electronics unit, e.g., by one or more of a soldering connection, a bonding connection, a plug, a clamping connection or the like. The electronics unit specifically may be used and/or configured as a transmitter for transmitting measurement data to at least one external device, such as to at least one receiver, e.g., wirelessly.

The electronics unit is electronically connected to the analyte sensor. Thus, an electrical connection exits between the analyte sensor and the electronics unit. Thus, an electrical signal, such as an electrical current and/or an electric voltage, may be transmitted via the electronic connection from the analyte sensor to the electronics unit. Via the electrical connection, the electronics unit may interact with the analyte sensor for performing at least one electrochemical measurement. The electrical connection specifically, as outlined above, may be established by at least one connection portion of the analyte sensor protruding into a housing of the electronics unit. The functional module may be preassembled in the sense that, the electronics unit is already electrically connected to the analyte sensor when the functional module is received in the housing, with the protective cap being connected to the housing. Specifically, the electronics unit may be irreversibly electrically connected to the analyte sensor. Thus, specifically, no need for assembly of the electronics unit and the analyte sensor is given, since, in the preassembled functional module, the electronics unit and the analyte sensor are already connected, electrically and optionally also mechanically.

The term “insertion component” as used herein is a broad term and is to be given its ordinary and customary meaning to a person of ordinary skill in the art and is not to be limited to a special or customized meaning. The term specifically may refer, without limitation, to an element or a combination of elements which are configured for inserting at least one component into a body tissue of a user, e.g., transcutaneously or subcutaneously. Thus, specifically, the at least one insertion component may be or may comprise at least one of an insertion cannula or an insertion needle, with a tip or sharp configured for piercing the skin of the user and further, optionally, with at least one slot configured for receiving at least a part of the analyte sensor. The insertion component may comprise further elements, such as at least one holder for manipulating or holding the insertion component such as the insertion cannula and/or the insertion needle.

Within the context of this disclosure, the term “insertion cannula” is a broad term and is to be given its ordinary and customary meaning to a person of ordinary skill in the art. An insertion cannula is a usually a hollow needle which may be at least partially slotted. The analyte sensor may then typically be received within the cannula, meaning within the hollow part.

Within the context of this disclosure, the term “insertion needle” is a broad term and is to be given its ordinary and customary meaning to a person of ordinary skill in the art. An insertion needle is typically a compact needle, specifically without a slot and without any hollow parts. The analyte sensor may then typically be received on an outer surface of the insertion needle.

The term “inserting the analyte sensor into a body tissue of the user” as used herein is a broad term and is to be given its ordinary and customary meaning to a person of ordinary skill in the art and is not to be limited to a special or customized meaning. The term specifically may refer, without limitation, to an action of one or more of transcutaneously or subcutaneously applying the analyte senor to a body tissue of the user. Specifically, the analyte sensor may fully or partially be inserted into a body tissue of the user in such a way that the analyte sensor may detect the at least one analyte in the body tissue of the user. The action of inserting the analyte sensor may be performed by the user himself, specifically by using the medical system according to this disclosure, such as any one of the embodiments disclosed herein and/or disclosed in further detail below.

The term “protective cap” as used herein is a broad term and is to be given its ordinary and customary meaning to a person of ordinary skill in the art and is not to be limited to a special or customized meaning. The term specifically may refer, without limitation, to an element configured for at least partially covering at least one other device, component or element, thereby providing at least partial protection against mechanical and/or environmental influences. The protective cap specifically may be fully or partially made of at least one rigid material, such as of at least one plastic material and/or at least one metallic material. The protective cap specifically may have an opening which is configured to be directed towards the housing of the medical system. The protective cap specifically may be made essentially rotationally symmetric, e.g., by having an axial rotational symmetry about an axis such as a cylinder axis or axis of extension. The protective cap, as an example, may be designed as a cylinder, a hemisphere or as a dome.

The protective cap, as an example, may be connected to the housing by at least one of a form-fit or a force-fit connection or even as an integral component of the housing as a break-away-part. For example, a rim of the protective cap may be pushed over a rim of the housing or vice a versa. Thus, the protective cap may have a circular, oval or polygonal rim which fits tightly over a rim of the housing having a corresponding shape, or vice a versa. As another example, the protective cap may be connected to the housing via a threaded joint. Thus, both, the housing and the protective cap may have a thread which are compatible with each other. There may be an overlap region in the connected state, in which the protective cap overlaps with the housing or vice a versa. As another example the protective cap is part of the housing connected by a thin-walled portion with limited strength.

The term “removable” as used herein is a broad term and is to be given its ordinary and customary meaning to a person of ordinary skill in the art and is not to be limited to a special or customized meaning. The term specifically may refer, without limitation, to a characteristic of a device, element or component of the medical system of being detachable from the medical system. Specifically, a removable component, such as the protective cap, may be detached by the action of a user. For example, the protective cap may be removed by the user from the housing of the medical system prior to application. The protective cap may be removed from the housing by at least one of: pulling the protective cap off the housing; turning the protective cap off the housing.

The term “covering the preassemble functional module” as used herein is a broad term and is to be given its ordinary and customary meaning to a person of ordinary skill in the art and is not to be limited to a special or customized meaning. The term specifically may refer, without limitation, to a fully or partially shielding of the functional module from environmental influences, such as mechanical and/or chemical influences. The preassembled functional module may be fully covered by the removable protective cap in a state prior to application. In this state, the preassembled functional module may not be visible from the outside of the medical system. Thus, the preassembled functional module may be protected against mechanical and/or environmental influences, for example, humidity, by being covered by the protective cap. In case, the medical system may be used, the removable protective cap is detached from the housing and, thus, the insertion component may be revealed.

The term “sterility cap” as used herein is a broad term and is to be given its ordinary and customary meaning to a person of ordinary skill in the art and is not to be limited to a special or customized meaning. The term specifically may refer, without limitation, to an element such as a cover which is configured for maintaining a sterile atmosphere in an interior space fully or partially surrounded by the element. Specifically, the sterility cap may provide a sealed interior space, wherein the sealed interior space may at least partially surround the analyte sensor and the insertion component. Further, the sterility cap, as an example, may be a rigid sterility cap, e.g., made of a rigid plastic material and/or a metallic material. The sterility cap, as an example, may have a rotational symmetry about an axis, such as an axis of extension, which, as an example, may be identical to a rotational symmetry axis of the protective cap and/or of a rotational symmetry axis of the housing. The rotational symmetry may be a continuous rotational symmetry, e.g., a symmetry with respect to a continuous rotation about the symmetry axis, and/or a discrete rotational symmetry, e.g., a symmetry with respect to a discrete rotation about the symmetry axis. For example, the sterility cap may comprise one or more notches forming an interlocking with the protective cap and, specifically, being configured for applying a torque to the sterility cap. It shall be noted, however, that other shapes without having a rotational symmetry are also feasible. Thus, as an example, the sterility cap may also have an asymmetric shape. An asymmetric shape, as an example, might simplify removing the sterility cap, e.g., by using an appropriately shaped tool or by hand. Generally, the sterility cap may have a prismatic shape. The sterility cap may have an elongated shape, with a length exceeding its diameter or equivalent diameter by at least a factor of 2, more preferably by at least a factor of 5. The sterility cap, as an example, may have a length of 5 to 20 mm, e.g., a length of 10 to 15 mm.

As outlined above, the sterility cap at least partially surrounds the insertion component, such as the insertion cannula and/or the insertion needle. The sterility cap may therefore provide a sterile interior space to the insertion cannula and/or the insertion needle. The sterility cap further comprises at least one sterility testing access, such as at least one septum and/or at least one multiple-step sealing. Thus, the sterility cap may provide access to the sterile components of the medical system, such as the insertion component and/or the analyte sensor.

The term “sterility testing access” as used herein is a broad term and is to be given its ordinary and customary meaning to a person of ordinary skill in the art and is not to be limited to a special or customized meaning. The term specifically may refer, without limitation, to an element configured for providing access to an interior space of the sterility cap in such a way that a contamination present outside the interior space may not be transferred into the interior space of the sterility cap. The sterility testing access comprises at least one of a septum and a multiple-step sealing, as will be described in further detail below.

The sterility testing access may be arranged at a distal end of the sterility cap, i.e., at an end facing away from the preassembled function module and/or from the electronics unit thereof. Thus, as outlined above, the sterility cap may have an elongated shape, such as a conical shape and/or a cylindrical shape. At one end, specifically the proximal end, the preassembled functional may be located, such that, as an example, the interior space of the sterility cap is sealed by the preassembled functional module, such as a base plate of the preassembled functional module. At an opposing end, such as the distal end, the sterility testing access may be located, comprising the septum and/or the multiple-step sealing.

The sterility testing access specifically may be arranged within an opening of the removable protective cap and, thus, may be accessible from an outer side of the medical system. Specifically, the sterility testing access may be accessible through the removable protective cap when the removable protective cap is connected to the housing. The sterility testing access may be invisible to the user of the medical system, for example, by being shielded by a removable seal covering the opening of the protective cap.

The term “septum” as used herein is a broad term and is to be given its ordinary and customary meaning to a person of ordinary skill in the art and is not to be limited to a special or customized meaning. The term specifically may refer, without limitation, to a sealing element made of an elastic partition configured for separating the sterile interior space of the sterility cap from the outside. The septum may be pierceable with a sharp element, such as a tip, a needle, a cannula or the like, and, thus, may enable the sharp element to penetrate through the septum and enter the interior space of the sterility cap. For example, a needle may be able to pierce the septum and, thus, may have access to the interior space of the sterility cap. As an example, the sterility cap may comprise a tubular sidewall, such as a cylindrical or conical side wall, which may be made of a rigid plastic material, wherein the tubular sidewall surrounds a sterility testing access opening, wherein the septum is located in the sterility testing access opening, wherein the septum is made of a deformable material, e.g., an elastic material, such as silicone rubber and/or other types of rubber.

The term “multiple-step sealing” as used herein is a broad term and is to be given its ordinary and customary meaning to a person of ordinary skill in the art and is not to be limited to a special or customized meaning. The term specifically may refer, without limitation, to a sealing element comprising a plurality of sealing steps which are configured for receiving and/or engaging with a removable closing element, such as a removable stopper, a removable cap, a removable shutter. The multiple-step sealing, as an example, may have a rotational symmetry about an axis which, as an example, may be identical to a rotational symmetry axis of the sterility cap, to a rotational symmetry axis of the protective cap and/or of a rotational symmetry axis of the housing. The plurality of sealing steps may have different equivalent diameters and, thus, the removable closing element may be received by a part of the sealing steps, such as by one, two or even more of the sealing steps. The multiple-step sealing may be configured for sealing the interior space of the sterility cap at one or more of the outer sealing steps, whereby the inner sealing steps are not in contact with the closing element. The closing element may seal the interior space of the sterility cap at a distal end of the sterility cap.

The multiple-step sealing may comprise a plurality of circumferential sealing elements having differing diameters or equivalent diameters and being disposed within the interior space of the sterility cap. For example, the multiple-step sealing may comprise a plurality of circumferential sealing elements with decreasing diameters or equivalent diameters from outside to inside of the sterility cap. The circumferential sealing elements may be disposed concentrically, specifically with respect to an axis of extension of the sterility cap. Further, the circumferential sealing elements may comprise at least one of: circumferential sealing lips; circumferential sealing edges; circumferential sealing shoulders; circumferential sealing angles. The circumferential sealing elements may comprise tubular sections within the sterility cap, the tubular sections having differing equivalent diameters.

The term “circumferential sealing elements” as used herein is a broad term and is to be given its ordinary and customary meaning to a person of ordinary skill in the art and is not to be limited to a special or customized meaning. The term specifically may refer, without limitation, to sealing elements surrounding the sterility testing access. Specifically, the circumferential sealing elements may be arranged concentrically with respect to an axis of symmetry and/or extension of the sterility cap. The circumferential sealing elements, as an example, may have a rotational symmetry about an axis which, as an example, may be identical to a rotational symmetry axis of one or more of the sterility cap, the protective cap and/or the housing. Further, at least part of the circumferential sealing elements may be configured for fitting tightly with at least one optional removable stopper closing the sterility testing access opening. For example, the circumferential sealing elements may comprise at least one of: circumferential sealing lips; circumferential sealing edges; circumferential sealing shoulders; circumferential sealing angles. The circumferential sealing elements may comprise tubular sections within the sterility cap, the tubular sections having differing equivalent diameters. As another example, the circumferential sealing elements may have decreasing equivalent diameters from outside to inside of the sterility testing access. Thus, the removable stopper of the sterility testing access opening may fit tightly to at least one outer circumferential sealing element, wherein the at least one inner circumferential sealing element may not be in contact with the removable stopper. Later on, for sterility testing, when the stopper is removed and a fluidic adapter is attached to the sterility testing access, the fluidic adapter may be in contact with the inner circumferential sealing element, only, thereby avoiding carrying microbial contaminations into the interior space.

Further, the sterility cap may comprise a tubular sidewall surrounding a sterility testing access opening. The sterility testing access may be disposed within the sterility testing access opening. The sterility testing access opening, as an example, may be fully or partially closed by the septum. As another example, the multiple-step sealing may comprise multiple circumferential sealing elements disposed within the sterility testing access opening. Thus, the sterility testing access opening may be closed by at least one of the septum and the removable stopper.

The protective cap specifically may comprise an opening, wherein, as an example, the sterility cap is arranged within the opening, specifically such that the sterility cap fits into the opening of the protective cap. Thus, the sterility cap and the protective cap may interact in a lock and key-principle, by using an appropriate shape of the opening. Further, the opening may also, as will be explained in further detail below, provide guidance for a fluidic adapter and/or may protect a fluidic adapter against twisting. Further, the sterility cap, specifically the sterility testing access of the sterility cap, may be accessible from an outer side of the medical system. Specifically, the sterility cap may be accessible through the opening in the removable protective cap, more specifically through the removable protective cap when the removable protective cap is connected to the housing. Thus, the opening of the protective cap may form an access to the sterility cap, specifically from an outer side of the medical system. The access to the sterility cap may be sealed by a removable seal, specifically by a removable liner, more specifically by a removable liner covering an opening in the protective cap through which the sterility cap may be accessible after removing the removable seal. Further, as discussed above, the opening of the protective cap may be configured for guiding the fluidic adapter to the sterility testing access, specifically, when the fluidic adapter is attached to the sterility testing access. The opening may guide the fluidic adapter in such a way that a distortion, such as a twisting or a rotational motion, of the protective cap is prevented when the fluidic adapter is attached to the sterility testing access.

As outlined above, the removable protective cap may comprise an opening, wherein the removable protective cap, within the opening, may surround the sterility cap. The opening may be located within a distal surface of the removable protective cap, such as a surface facing a skin site of a user before inserting the analyte sensor into a body tissue of the user by using the medical system, specifically by using the insertion component of the medical system.

Further, the removable protective cap may be mechanically interlocked with the sterility cap within the opening. Thus, when the removable protective cap is removed from the housing, such as in preparation for insertion, the sterility cap may be removed, too.

Thus, the sterility cap may be engaged with the removable protective cap, such that, when the protective cap is removed from the housing, the sterility cap may be removed from the preassembled functional module. For this purpose, the sterility cap may be mechanically interlocked with the removable protective cap. The mechanical interlock of the sterility cap with the removable protective cap may be or may comprise a predetermined breaking point, e.g., a breaking point of the sterility cap with a base plate of the preassembled functional module. Thus, generally, in between the sterility cap and the base plate of the preassembled functional module, at least one predetermined breaking point may be provided, such as a circumferential breaking line. The at least one breaking point, as an example, may comprise a weakening in a wall of the sterility cap, such as a circumferential groove. Thus, in preparation for using the medical system, e.g., in preparation for insertion, the sterility cap surrounding the insertion component may be removed together with the protective cap.

Further, the sterility cap may have at least one side in common with the protective cap. The sterility testing access may be located at the side which is in common with the protective cap. The sterility cap may have a tubular shape, wherein the sterility testing access may be located at an end of the sterility cap, specifically at a distal end of the sterility cap. The distal end of the sterility cap may refer to the end of the sterility cap pointing towards the skin of the user of the medical system.

Further, the preassembled functional module may comprise at least one base plate. As outlined above, the medical system comprises the housing and the removable protective cap connected to the housing. Thus, the housing and the removable protective cap connected to the housing may, in combination, form a system housing which encases the preassembled functional module as well as, optionally, other components of the medical system. The preassembled functional module, as such, may comprise the base plate as well as, as will be outlined in further detail below, at least one cover element, such that the base plate and the cover element form at least one module housing, wherein the module housing may encase one or more components of the functional module, such as the electronics unit.

The term “base plate” as used herein is a broad term and is to be given its ordinary and customary meaning to a person of ordinary skill in the art and is not to be limited to a special or customized meaning. The term specifically may refer, without limitation, to a rigid element being capable of providing mechanical support for one or more other elements. The base plate specifically may have a flat shape, such as a shape of a disk. The base plate, however, may also comprise one or more supporting elements, such as one or more spacers, lugs, mounting protrusions or the like. Further, the base plate may comprise at least one connection element for engaging with at least one of: the electronics unit, the analyte sensor, the sterility cap, a cover element of the preassembled functional module. The base plate specifically may have an essentially flat lower sider and/or an essentially flat upper side.

The base plate may be configured for at least partially receiving the preassembled functional module, specifically the analyte sensor, the electronics unit and/or the insertion component. The sterility cap may be connected to the base plate. The sterility cap and the base plate, as an example, may be made in a single piece, such as by commonly molding these parts. As another example, the sterility cap and the base plate may be manufactured separately and may be connected by means of at least one of: a welding technique; a bonding technique; a soldering technique; an adhesive method; a press fit or a thread.

The analyte sensor and the insertion component may penetrate the base plate via at least one through hole from an upper side to a lower side, wherein the sterility cap, on the lower side, may surround the through hole. The lower side may refer to the side of the medical system facing towards the skin of the user, wherein the upper side may refer to the side facing towards the housing. For example, the insertion device and the analyte sensor may protrude from the through hole, specifically both on the upper and on the lower side of the though hole.

As outlined above, the analyte sensor may be received by the base plate. Thus, the analyte sensor may be mounted to the base plate by at least one sensor fixation element, specifically by a sensor fixation element sealing the through hole. For example, the sensor fixation element may be comprised by the base plate on the upper side of the base plate and, thus, may seal the through hole on the upper side. The sensor fixation element may further be configured for connecting at least one of the analyte sensor and/or the insertion component to at least one driving actuator, as will be outlined in further detail below.

Further, the preassembled functional module may comprise at least one cover element. The term “cover element” as used herein is a broad term and is to be given its ordinary and customary meaning to a person of ordinary skill in the art and is not to be limited to a special or customized meaning. The term specifically may refer, without limitation, to an element being capable of covering at least one other element, e.g., by fully or partially surrounding the at least one other element. The cover element specifically may comprise at least one rigid cover element, such as a rigid cover element made of a rigid plastic material and/or a rigid metallic material. The cover element specifically may comprise a rigid cap. The cover element may cover the electronics unit and at least part of the analyte sensor. The cover element may also be referred to as electronics unit housing. Specifically, the preassembled functional module may comprise a cover element being disposed on the base plate on the upper side. As an example, the electronics unit may be disposed in a space formed by the base plate and the cover element. For example, the cover element may be connected to the base plate and, thus, may fix the electronics unit and at least part of the analyte sensor to the base plate.

As outlined above, the sterility cap may provide a sealed interior space. The analyte sensor and the insertion component may at least partially be located within the sealed interior space. The sealed interior space may be sealed in an airtight fashion. The sealed interior space may be sterilized. The sealed interior space may have a volume of no more than 1 cm³, specifically a volume of 0.01 to 1.0 cm³.

Further, the medical system may comprise at least one driving actuator for driving the insertion component into a body tissue of the user. The term “driving actuator” as used herein is a broad term and is to be given its ordinary and customary meaning to a person of ordinary skill in the art and is not to be limited to a special or customized meaning. The term specifically may refer, without limitation, to an arbitrary device which is configured for directly or indirectly inserting at least one insertable element into a body tissue, e.g., by transferring a driving force onto the insertable element. Specifically, the driving force may be transferred to the insertion component, such as the insertion cannula or the insertion needle. The driving actuator may, thus, comprise at least one mechanical actuator for transferring one or both of a manual force or a force provided by a mechanical element such as a spring onto the insertion component. The driving actuator may, thus, comprise at least one mechanical device which is configured for driving forward the insertable element or the insertion component such as the insertion cannula and/or the insertion needle into the body tissue. The driving actuator, as an example, may comprise at least one slider connected to the insertion component or a holder for the insertion component and may be configured for performing a forward linear motion in an insertion direction and, optionally, a backward linear motion in an opposite direction. As an example, the slider may be driven by at least one spring element, which may be pretensioned or biased in the forward direction or in the backward direction. Additionally or alternatively, the slider may be connected to at least one actuation button which may be pushed by the user, thereby driving the slider in the forward direction.

The driving actuator may be configured for driving the insertion component into the body tissue and, optionally, backwards again after insertion of the analyte sensor. The backward motion, as an example, for retracting the insertion component such as the insertion cannula and/or the insertion needle from the body tissue, may be driven by a return spring or by a motion inverter. Generally, the driving actuator may be configured for advancing the insertion component after removal of the protective cap from the housing and for inserting the analyte sensor into the body tissue.

The housing of the medical system may further comprise a base part. The base part may refer to the part of the medical system being attached or positioned to the skin of the user. Specifically, the base part may be attached to the skin of the user when the insertion component inserts the analyte sensor into the body tissue of the user. The base part may be covered by the removable protective cap and may be revealed prior to application when the removable protective cap is removed by the user. Further, the driving actuator may comprise a plunger movable with respect to the base part, the plunger being connected to the insertion component.

Thus, as outlined above, the housing and the removable protective cap connected to the housing may, in combination, form a system housing which encases the preassembled functional module as well as, optionally, other components of the medical system. For preparing the medical system for use, the user may remove the removable protective cap from the housing. Thereby, the base part of the housing may be uncovered and may, thus, be positioned on the skin of the user, in preparation for insertion.

The preassembled functional module may further comprise at least one electrical energy storage device, specifically at least one battery. The electrical energy storage device may be configured for storing electrical energy. The electrical energy storage device may be configured for supplying electrical energy to the electronics unit when the medical system may be used by the user. Prior to application of the medical system, the electronics unit, specifically the microcontroller and/or the processor of the electronics unit, may be in a standby mode and, thus, may be activated when the medical system may be used by the user.

In a further aspect of this disclosure, a method of sterility testing of a medical system is disclosed. The medical system comprises a preassembled functional module. The preassembled functional module comprises an analyte sensor for detecting at least one analyte in a body fluid of a user, and an electronics unit electrically connected to the analyte sensor and an insertion component for inserting the analyte sensor into a body tissue of the user. The preassembled functional module further comprises at least one sterility cap at least partially surrounding the insertion component and optionally at least a part of the analyte sensor. As an example, the medical system may be embodied according to any one of the embodiments disclosed above and/or according to any one of the embodiments disclosed in further detail below. Thus, for possible definitions and options, reference may be made to the disclosure of the medical system according to this disclosure.

The method comprises the following steps which specifically may be performed in the given order. Further, it is also possible to perform one or more of the method steps once or repeatedly. Further, it is possible to perform one or more of the method steps simultaneously or in a timely overlapping fashion. The method may comprise further method steps which are not listed.

The method comprises:
A) inserting a rinsing liquid into an interior space of the sterility cap; and
B) microbial testing of the rinsing liquid.

The method may further comprise at least one step of removing the rinsing liquid from the interior space of the sterility cap. This step specifically may be performed before performing step B). Thus, the microbial testing in step B) may be performed by using rinsing liquid which previously was inside the interior space and which, subsequently, was been removed from the interior space for the purpose of testing. Additionally or alternatively, the rinsing liquid may also still be in fluidic contact or fluidic exchange with the interior space or with a part of the liquid still inside the interior space during microbial testing. Examples will be given below.

The term “sterility testing” as used herein is a broad term and is to be given its ordinary and customary meaning to a person of ordinary skill in the art and is not to be limited to a special or customized meaning. The term specifically may refer, without limitation, to the process of identifying and/or monitoring a degree of microbial contamination of an element, including a monitoring of a change of the degree of microbial contamination of the element. Specifically, the sterility testing may comprise an arbitrary process of one or more of assessing or verifying the sterility of the medical system. Specifically, the sterility testing may be a direct or an indirect method to test or verify the sterility of the medical system. The sterility of the medical system may be evaluated in terms of contaminations, specifically in terms of microbial contaminations such as bacteria, viruses and/or other microorganism, present in the medical system. For example, the sterility testing may comprise rinsing of the medical system with a rinsing liquid and subsequent incubating of the rinsing liquid. The rinsing liquid may be brought into contact with the parts of the medical system to be tested. Thus, the rinsing liquid may incorporate possible contaminations from the medical system. By incubating the rinsing liquid, possible contaminations of the rinsing liquid may be determined. Thus, the sterility testing may be an indirect method, whereby a possible contamination of the medical system may be determined apart from the medical system.

The term “rinsing liquid” as used herein is a broad term and is to be given its ordinary and customary meaning to a person of ordinary skill in the art and is not to be limited to a special or customized meaning. The term specifically may refer, without limitation, to an arbitrary liquid, such as a pure liquid, or a liquid mixture or a suspension, which is configured for receiving microbial contaminations and/or which is configured for being incubated.

A pure liquid may be one single liquid or a solution of at least one liquid and another component, such as a solid. A liquid mixture may be a solution of at least two liquids or an emulsion of two liquids.

The rinsing liquid specifically may comprise a nutrient solution. The rinsing liquid may be a sterile liquid, specifically prior to application, which may be configured for sterility testing of the medical system. For example, the rinsing liquid may be a solution containing one or more nutrients dissolved in an aqueous liquid.

The term “inserting the rinsing liquid” as used herein is a broad term and is to be given its ordinary and customary meaning to a person of ordinary skill in the art and is not to be limited to a special or customized meaning. The term specifically may refer, without limitation, to an action of at least partially introducing the rinsing liquid into the interior space of the sterility cap. Specifically, the rinsing liquid may be inserted into the interior space of the sterility cap through the sterility testing access, more specifically through the sterility testing access opening. For inserting the rinsing liquid, the sterility testing access may comprise at least one of the septum and/or the multiple-step sealing, as outlined above. Thus, inserting the rinsing may comprise attaching the fluidic adapter to the sterility testing access. The rinsing liquid may be inserted into the interior space of the sterility cap by using a fluidic adapter attached to the sterility testing access. Further, inserting the rinsing liquid into the interior space of the sterility cap may be performed in such a way that the rinsing liquid may contact the sterile parts, specifically at least part of the insertion component and/or the analyte sensor. Thus, the rinsing liquid may incorporate possible contaminations of the sterile parts, specifically of the insertion component and/or the analyte sensor.

The term “removing the rinsing liquid” as used herein is a broad term and is to be given its ordinary and customary meaning to a person of ordinary skill in the art and is not to be limited to a special or customized meaning. The term specifically may refer, without limitation, to an action of fully or partially retracting the inserted rinsing liquid from the interior space of the sterility cap. Specifically, removing the rinsing liquid may comprise an uptake and a subsequent sealing of the rinsing liquid. For example, the inserted rinsing liquid may be removed by using a fluidic adapter attached to the sterility testing access. As another example, inserting and removing the rinsing liquid may be done by constant flushing the interior space with the rinsing liquid. The inserting the rinsing liquid into the interior space and the removing of the rinsing liquid from the interior space, together, may comprise flushing the interior space with the rinsing liquid and/or guiding the rinsing liquid through at least a part of the interior space. The volume of the rinsing liquid may, thus, exceed the volume of the interior space.

The term “microbial testing” as used herein is a broad term and is to be given its ordinary and customary meaning to a person of ordinary skill in the art and is not to be limited to a special or customized meaning. The term specifically may refer, without limitation, to an action of controlling the rinsing liquid for microbial contaminations. The microbial testing may comprise incubating the rinsing liquid. Specifically, the microbial testing of the rinsing liquid may be done apart from the medical system, such as in an incubator. The result of the microbial testing may refer to an assessment of the sterility of the medical system, specifically whether the interior space of the medical system is sterile or not. As an example, the microbial testing may also be performed prior to step C), that is removing the rinsing liquid from the interior space of the sterility cap. Thus, the rinsing liquid may be incubated while the fluidic adapter is attached to the sterility testing access.

As an example, in step A), a reservoir of the rinsing liquid, such as a reservoir of a nutrient solution, may be attached to the sterility cap, such as a reservoir having a volume of at least 3 ml or even of at least 5 ml. At least a part of the content of the reservoir may be inserted into the interior space of the sterility cap, such as by completely filling the interior space of the sterility cap with the rinsing liquid. Therein, a fluidic contact between the rinsing liquid inside the interior space and the reservoir may be established and/or maintained, such that microbial contaminations from the interior space may migrate or diffuse into the reservoir, by itself or with a part of the rinsing liquid. Step B) may then comprise microbial testing such as breeding of the rinsing liquid, e.g., the nutrient solution. The breeding may result in a detectable change, such as one or more of a color change, a change in transparency or a visible formation of turbidity in the rinsing fluid which may be detected visually and/or by using a sensor. In case the change is detected, the microbial testing may indicate that the interior space of the sterility cap was contaminated. Specifically, the total amount of the rinsing liquid may exceed the volume of the interior space, in order to provide a sufficient amount of rinsing liquid for detecting the detectable change in case a microbial contamination is present.

Thus, the method of sterility testing may comprise:
docking a reservoir with a rinsing liquid, such as a nutrient solution or a nutrition liquid, to the sterility cap;
inserting at least a portion of the rinsing liquid into the sterility cap, specifically into the interior space of the sterility cap (step A));
incubating the arrangement and microbial testing of the rinsing liquid (step B).

The rinsing liquid may be applied to the interior space of the sterility cap through the sterility testing access of the sterility cap. The sterility testing access may comprise the at least one septum, as an example, whereby the rinsing liquid may be applied to the interior space of the sterility cap by piercing the septum with a cannula and introducing the rinsing liquid through said cannula. As another example, the sterility testing access may comprise the multiple-step sealing, whereby the rinsing liquid may be applied to the interior space of the sterility cap by using a fluid adapter, as will be described in detail below.

Further, the microbial testing of the rinsing liquid may comprise incubating the rinsing liquid. Incubating the rinsing liquid may be done in step C) of the method of sterility testing.

As outlined above, the preassembled functional module may be at least partially received in the housing at least partially surrounding the preassembled functional module. In step A), the rinsing liquid may be applied to the interior space of the sterility cap, only, without contacting the housing. Thus, the rinsing liquid may be applied to the interior space of the sterility cap only, without contacting the sterility testing access.

The method of sterility testing may comprise a medical system, wherein the medical system may be a medical system according to this disclosure, such as according to any one of the embodiments disclosed above and/or according to any one of the embodiments disclosed in further detail below referring to a medical system.

As outlined above, the sterility cap may have at least one sterility testing access. Step A) may comprise attaching at least one fluidic adapter to the sterility testing access. The fluidic adapter may also be sterilized or may be sterilizable prior to application.

The terms “sterilizing” or, a synonymously used, “sterilization” as used herein are broad terms and are to be given their ordinary and customary meaning to a person of ordinary skill in the art and are not to be limited to a special or customized meaning. The terms specifically may refer, without limitation, to an action of reducing or even eliminating all forms of life and biological agents present, such as in or on an object. Specifically, sterilization may refer to any process that eliminates, removes, kills, or deactivates all forms of life, in particular microorganisms and/or other biological agents like prions, such as in a specific surface, object or fluid, for example, food or biological culture media. Sterilization can be achieved through various means, including heat, chemicals, irradiation, high pressure, and filtration. Specifically, the sterilization may comprise sterilization by irradiation with e-beams and/or electromagnetic radiation, such as gamma rays. The terms “sterilized” and “sterile,” correspondingly, may refer to an article, object or surface which has undergone the process of sterilization.

For example, the sterility testing access may comprise at least one multiple-step sealing, wherein step A) may comprises removing at least one stopper from the sterility testing access. The stopper may be in contact with a first-step sealing of the multiple-step sealing. Step A) may further comprise attaching the fluidic adapter to the sterility testing access, wherein the fluidic adapter may be in contact with a second-step sealing of the multiple-step sealing. The first-step sealing may have a larger equivalent diameter than the second-step sealing. Thus, the fluidic adapter may be in contact only with the second-step sealing. The second-step sealing may be disposed closer to the insertion component than the first-step sealing. As another example, the fluidic adapter may be a syringe adapter. The syringe adapter may comprise at least one cannula configured for piercing the septum and introducing the rinsing liquid into the interior space of the sterility cap.

In a further aspect of this disclosure, a method of providing the medical system according to this disclosure is proposed, such as according to any one of the embodiments disclosed above and/or according to any one of the embodiments disclosed in further detail below.

The method comprises the following steps which specifically may be performed in the given order. It shall be noted, however, that a different order is also possible. Further, it is also possible to perform one or more of the method steps once or repeatedly. Further, it is possible to perform one or more of the method steps simultaneously or in a timely overlapping fashion. The method may comprise further method steps which are not listed.

The method comprises:
I. assembling a part of the preassembled functional module, the part of the preassembled functional module comprising the analyte sensor and the insertion component as well as the sterility cap at least partially surrounding the insertion component;
II. sterilizing the part of the preassembled functional module, specifically by using one or more of e-beam or gamma sterilization;
III. assembling the preassembled functional module, the assembling comprising electrically connecting the electronics unit to the analyte sensor;
IV. assembling the medical system, the assembling comprising receiving the preassembled functional module in the housing and connecting the removable protective cap to the housing.

Steps I. and II. of the method of providing the medical system may comprise an assembling of the sterile parts of the medical system. The sterile parts may comprise at least one or more of the analyte sensor, the insertion component, the base plate, the sensor fixation element and/or the sterility cap. The sterilization in step I. specifically may take place after assembling the parts to be sterilized, such as a subgroup comprising the analyte sensor, the insertion component, the sterility cap, the base plate and the sensor fixation element.

The sterile parts may undergo further processing. Thus, in steps III. and IV. of the method of providing the medical system, the assembling may include assembling sterile and non-sterile parts of the medical system. Thus, in step III., at least one sterilized subgroup of the preassembled functional module, such as a subgroup assembled in step I. and sterilized in step II., e.g., a subgroup comprising the analyte sensor, the insertion component and the sterility cap, may be assembled with one or more non-sterilized parts of the preassembled functional module, such as the electronics unit and, optionally, the cover element. The non-sterile parts may comprise at least one or more of the electronics unit, the housing, the removable protective cap, the base plate, the base part, the cover element, the driving actuator and/or the electrical energy storage device. Thus, the method of providing the medical system may comprise an assembly process wherein sterile and non-sterile part may be processed.

The method may further comprise:
V. storing the medical system.

Thus, as an example, the medical system may be transferred to at least one storage facility, wherein the medical system is stored, such as for a storage time of, e.g., more than one day, more than one week or even more than one month.

The method may further comprise, specifically after storing the medical system in accordance with step V.:
VI. sterility testing of the medical system by using the method of sterility testing of a medical system according to this disclosure, such as according to any one of the embodiments disclosed above and/or according to any one of the embodiments disclosed in further detail below.

In step VI. of the method of providing the medical system, only few selected medical systems may be subjected to sterility testing. The medical system subjected to sterility testing may be selected on a random basis. The selection may be done in such a way, that the sterility testing of the few selected medical systems may allow for verifying the sterility of all assembled medical systems. For example, the percentage of medical systems subjected to sterility testing may be less than 1%, e.g., 0.1% or even less.

The methods and devices according to this disclosure provide a large number of advantages over known methods and devices. Specifically, the medical system according to this disclosure may provide for a fully integrated device for detecting at least one analyte in a body fluid of a user. The medical system may specifically be configured for continuous monitoring of glucose in a body tissue of a user. Further, the medical system comprises the preassembled functional module received in the housing and the removable protective cap. This preassembly specifically may be a factory preassembly of the medical system. Therefore, the medical system may be ready for use prior to unpacking the medical system by the user. Thus, the medical system according to this disclosure may provide an easy and user-friendly method for inserting an analyte sensor and subsequently continuous monitoring of at least one analyte in a body tissue of a user.

Further, the at least one sterility testing access comprised by the sterility cap of the medical system enables the manufacturer to test the sterile interior space of the medical system. Specifically, the at least one septum and/or the at least one multiple-step sealing may facilitate access to the sealed interior space of the medical system. By using a fluidic adapter, for example, a syringe, the rinsing fluid, e.g., a nutrient solution, may be applied to the interior space of the sterility cap without disassembling the medical system. This minimizes the risk of contamination in case the sterility of the sealed interior space shall be tested. Thus, the inventive method and the inventive devices may allow for sterility testing of the medical system to make sure that during the assembly of the medical system the sterile parts, such as the insertion component and the analyte sensor inside the interior space of the sterility cap were not damaged.

The medical system comprises the preassembled functional module comprising the analyte sensor and the insertion component which may be applied to the skin of a user. The insertion component inserts the analyte sensor into a body tissue of the user, for example, into the subcutaneous tissue of the user. Thus, the analyte sensor may be guided to the body tissue by the insertion component, such as the insertion needle and/or the insertion cannula. Prior to application, the sterility cap at least partially surrounding the insertion component may have to be removed. After inserting the analyte sensor into the body tissue of the user, the insertion component may be removed from the body tissue and the analyte sensor may be ready for use.

The preassembled functional module may comprise components to be inserted into the body tissue of the user which have to be sterile prior to application. The medical system may further comprise other functional units, such as the electronics unit, the electrical energy storage device and/or an adhesive plaster by which the medical system or a part thereof, such as the preassembled functional module, specifically the base plate thereof, may be attached to the skin of the user. The other functional units may be damaged when being sterilized, specifically by e-beam and/or gamma sterilization, which may be avoided by assembling the preassembled functional module from at least one sterilized part or subgroup and at least one non-sterilized part or subgroup, wherein the latter may comprise the sensitive components prone to damage during sterilization.

Further, the preassembled functional module may comprise a sealed interior space at least partially surrounding the insertion component and/or the analyte sensor. The insertion component and the analyte sensor may be sterilized before being assembled to the medical system. The preassembled functional module may be received in the housing of the medical system in such a way that a user may be able to use the medical system and insert the insertion component into the body tissue. After assembly of the medical system, the sealed interior space may not be accessible anymore. Thus, a method for sterility testing of the medical system may comprise the insertion of a rinsing liquid into the interior space of the sterility cap, whereby a possible contamination may be incorporated by the rinsing liquid. The rinsing liquid may be incubated, e.g., apart from the device to be tested and/or while still being fluidically connected to the device, and a possible contamination may be determined. For possible examples of the sterility testing, reference may be made to the examples given above or given in further detail below.

As an example, the method of sterility testing may comprise attaching the fluidic adapter to the sterility testing access and inserting at least a part of the rinsing liquid into the interior space of the sterility cap. Thus, at least a part of the rinsing liquid may remain outside the interior space of the sterility cap, but may fluidically be in contact with the rinsing liquid inside the interior space. In this state, step B) of the method may be performed. Thus, the rinsing liquid may be incubated with the fluidic adapter attached to the sterility testing access, wherein the rinsing liquid outside the interior space may be in fluidic contact with the sterile parts inside the interior space of the sterility cap. Alternatively, however, the rinsing liquid may also be removed from the interior space, e.g., by disconnecting the fluidic adapter from the sterility testing access, before microbial testing of the rinsing liquid.

In order to insert the rinsing liquid into the interior space of the sterility cap, the sterility cap comprises the at least one sterility testing access, such as the at least one septum and/or the at least one multiple-step sealing. Before inserting the rinsing liquid, the outer part of the sterility testing access may be sterilized, specifically by means which do not affect the interior of the sterile compartment, e.g., H₂O₂. Thus, the rinsing liquid may be inserted by the sterile fluidic adapter, such as a sterile cannula, and may be removed after being exposed to the interior space for a predetermined time span. Subsequently, the rinsing liquid may be microbially tested, for example, by incubating the rinsing liquid.

The sterility testing access may be accessible from an outer side of the medical system via the opening in the protective cap. The opening may be sealed by a removable seal, such as a removable liner, whereby the removable seal may hide the opening of the protective cap to the user. For sterility testing of the medical system, the removable seal may be removed and the method of sterility testing may be performed without further ado.

The sterility testing access may comprise the at least one multi-step sealing, specifically a two-step-sealing. The sterility testing access may be sealed by the removable stopper, specifically sealing the sterility testing access at an outer first-step sealing of the multi-step sealing. The first-step sealing may be exposed to a possible contamination from the outside of the medical system.

The method of sterility testing of the medical system may comprise removing the removable stopper and attaching the fluidic adapter to the sterility testing access. The fluidic adapter may comprise a syringe for inserting and removing the rinsing liquid. The fluidic adapter may be in contact with the second-step sealing of the multi-step sealing. The first-step sealing may have a larger equivalent diameter than the second-step sealing. The second-step sealing of the multiple-step sealing may be a sterile part of the medical system. Thus, the risk of a possible contamination during sterility testing from the outer side of the medical system may be minimized.

Summarizing and without excluding further possible embodiments, the following embodiments may be envisaged.

Embodiment 1: A medical system comprising:
i) a housing;
ii) a preassembled functional module received in the housing, the preassembled functional module comprising
   a. an analyte sensor for detecting at least one analyte in a body fluid of a user;
   b. an electronics unit electrically connected to the analyte sensor; and
   c. an insertion component for inserting the analyte sensor into a body tissue of the user, specifically at least one of an insertion cannula and an insertion needle; and
iii) at least one removable protective cap connected to the housing, covering the preassembled functional module,
   wherein the preassembled functional module further comprises at least one sterility cap, which at least partially surrounds the insertion component, specifically the insertion component and at least a part of the analyte sensor, wherein the at least one sterility cap is at least partially surrounded by the protective cap, specifically arranged at least partially within the protective cap, wherein the sterility cap comprises at least one sterility testing access, the sterility testing access comprising at least one of a septum and a multiple-step sealing, specifically a two-step sealing.

Embodiment 2: The medical system according to the preceding embodiment, wherein the multiple-step sealing comprises a plurality of circumferential sealing elements having differing equivalent diameters, the circumferential sealing elements being disposed within an interior space of the sterility cap.

Embodiment 3: The medical system according to the preceding embodiment, wherein the circumferential sealing elements are disposed concentrically, specifically with respect to an axis of extension of the sterility cap.

Embodiment 4: The medical system according to any one of the two preceding embodiments, wherein the circumferential sealing elements comprise at least one of: circumferential sealing lips; circumferential sealing edges; circumferential sealing shoulders; circumferential sealing angles.

Embodiment 5: The medical system according to any one of the three preceding embodiments, wherein the circumferential sealing elements comprise tubular sections within the sterility cap, the tubular sections having differing equivalent diameters.

Embodiment 6: The medical system according to any one of the preceding embodiments, wherein the sterility cap comprises a tubular sidewall surrounding a sterility testing access opening.

Embodiment 7: The medical system according to the preceding embodiment, wherein the sterility testing access is disposed within the sterility testing access opening.

Embodiment 8: The medical system according to any one of the two preceding embodiments, wherein the sterility testing access opening is fully or partially closed by the septum.

Embodiment 9: The medical system according to any one of the three preceding embodiments, wherein the multiple-step sealing comprises multiple circumferential sealing elements disposed within the sterility testing access opening.

Embodiment 10: The medical system according to any one of the four preceding embodiments, wherein the sterility testing access opening is closed by at least one of the septum and a removable stopper.

Embodiment 11: The medical system according to any one of the preceding embodiments, wherein the protective cap comprises an opening.

Embodiment 12: The medical system according to any one of the preceding embodiments, wherein the sterility cap, specifically the sterility testing access of the sterility cap, is accessible from an outer side of the medical system, specifically through the removable protective cap, more specifically through the removable protective cap when the removable protective cap is connected to the housing.

Embodiment 13: The medical system according to the preceding embodiment, wherein an access to the sterility cap is sealed by a removable seal, specifically by a removable liner, more specifically by a removable liner covering an opening in the protective cap through which the sterility cap is accessible after removing the removable seal.

Embodiment 14: The medical system according to any one of the preceding embodiments, wherein the removable protective cap comprises an opening, wherein the removable protective cap, within the opening, surrounds the sterility cap.

Embodiment 15: The medical system according to the preceding embodiment, wherein the opening is located within a distal surface of the removable protective cap.

Embodiment 16: The medical system according to any one of the two preceding embodiments, wherein the removable protective cap is mechanically interlocked with the sterility cap within the opening.

Embodiment 17: The medical system according to any one of the preceding embodiments, wherein the sterility cap is engaged with the removable protective cap, such that, when the protective cap is removed from the housing, the sterility cap is removed from the preassembled functional module.

Embodiment 18: The medical system according to any one of the preceding embodiments, wherein the sterility cap is mechanically interlocked with the removable protective cap.

Embodiment 19: The medical system according to any one of the preceding embodiments, wherein the sterility cap has at least one side in common with the protective cap.

Embodiment 20: The medical system according to the preceding embodiment, wherein the sterility testing access is located at the side which is in common with the protective cap.

Embodiment 21: The medical system according to any one of the preceding embodiments, wherein the sterility cap has a tubular shape, wherein the sterility testing access is located at an end of the sterility cap, specifically at a distal end of the sterility cap.

Embodiment 22: The medical system according to any one of the preceding embodiments, wherein the preassembled functional module further comprises at least one base plate, wherein the sterility cap is connected to the base plate.

Embodiment 23: The medical system according to the preceding embodiment, wherein the analyte sensor and the insertion component penetrate the base plate via at least one through hole from an upper side to a lower side, wherein the sterility cap, on the lower side, surrounds the through hole.

Embodiment 24: The medical system according to any one of the two preceding embodiments, wherein the analyte sensor is mounted to the base plate by at least one sensor fixation element, specifically by a sensor fixation element sealing the through hole.

Embodiment 25: The medical system according to any one of the preceding embodiments, wherein the preassembled functional module further comprises at least one cover element, wherein the cover element covers the electronics unit and at least part of the analyte sensor, more specifically a cover element being disposed on the base plate on the upper side.

Embodiment 26: The medical system according to any one of the preceding embodiments, wherein the sterility cap provides a sealed interior space, wherein the analyte sensor and the insertion component at least partially are located within the sealed interior space.

Embodiment 27: The medical system according to the preceding embodiment, wherein the sealed interior space is sterilized.

Embodiment 28: The medical system according to any one of the two preceding embodiments, wherein the sealed interior space has a volume of no more than 1 cm³, specifically a volume of 0.01 to 1.0 cm³.

Embodiment 29: The medical system according to any one of the preceding embodiments, wherein the medical system further comprises at least one driving actuator for driving the insertion component into a body tissue of the user.

Embodiment 30: The medical system according to the preceding embodiment, wherein the housing comprises a base part and wherein the driving actuator comprises a plunger movable with respect to the base part, the plunger being connected to the insertion component.

Embodiment 31: The medical system according to any one of the preceding embodiments, wherein the preassembled functional module further comprises at least one electrical energy storage device, specifically at least one battery.

Embodiment 32: A method of sterility testing of a medical system, the medical system comprising a preassembled functional module, the preassembled functional module comprising an analyte sensor for detecting at least one analyte in a body fluid of a user, and an electronics unit electrically connected to the analyte sensor and an insertion component for inserting the analyte sensor into a body tissue of the user, wherein the preassembled functional module further comprises at least one sterility cap at least partially surrounding the insertion component and optionally at least a part of the analyte sensor, wherein the method comprises:
A) inserting a rinsing liquid into an interior space of the sterility cap; and
B) microbial testing of the rinsing liquid.

Embodiment 33: The method according to the preceding embodiment, wherein the rinsing liquid is applied to the interior space of the sterility cap through a sterility testing access of the sterility cap.

Embodiment 34: The method according to any one of the two preceding embodiments, wherein the microbial testing of the rinsing liquid comprises incubating the rinsing liquid.

Embodiment 35: The method according to any one of the three preceding embodiments, wherein the preassembled functional module is at least partially received in a housing at least partially surrounding the preassembled functional module, wherein, in step A), the rinsing liquid is applied to the interior space of the sterility cap, only, without contacting the housing.

Embodiment 36: The method according to any one of the four preceding method embodiments, wherein the medical system is a medical system according to any one of embodiments 1 to 31.

Embodiment 37: The method according to any one of the five preceding method embodiments, wherein the sterility cap has at least one sterility testing access.

Embodiment 38: The method according to the preceding embodiment, wherein step A) comprises attaching at least one fluidic adapter to the sterility testing access.

Embodiment 39: The method according to the preceding embodiment, wherein the sterility testing access comprises at least one multiple-step sealing, wherein step A) comprises removing at least one stopper from the sterility testing access, the stopper being in contact with a first-step sealing of the multiple-step sealing, step A) further comprising attaching the fluidic adapter to the sterility testing access, wherein the fluidic adapter is in contact with a second-step sealing of the multiple-step sealing, wherein the first-step sealing has a larger equivalent diameter than the second-step sealing.

Embodiment 40: The method according to the preceding embodiment, wherein the second-step sealing is disposed closer to the insertion component than the first-step sealing.

Embodiment 41: The method according to any one of the three preceding embodiments, wherein the fluidic adapter is a syringe adapter.

Embodiment 42: A method of providing the medical system according to any one of embodiments 1 to 31, the method comprising:
I. assembling a part of the preassembled functional module, the part of the preassembled functional module comprising the analyte sensor and the insertion component as well as the sterility cap at least partially surrounding the insertion component;
II. sterilizing the part of the preassembled functional module, specifically by using one or more of e-beam or gamma sterilization;
III. assembling the preassembled functional module, the assembling comprising electrically connecting the electronics unit to the analyte sensor; and
IV. assembling the medical system, the assembling comprising receiving the preassembled functional module in the housing and connecting the removable protective cap to the housing.

Embodiment 43: The method according to the preceding embodiment, the method further comprising:
V. storing the medical system.

Embodiment 44: The method according to any one of the two preceding embodiments, the method further comprising:
VI. sterility testing of the medical system by using the method of sterility testing of a medical system according to any one of embodiments 32 to 41.

### BRIEF DESCRIPTION OF THE DRAWINGS

The above-mentioned aspects of exemplary embodiments will become more apparent and will be better understood by reference to the following description of the embodiments taken in conjunction with the accompanying drawings, wherein:

FIGS. 1a-b show an embodiment of a preassembled functional module in a cross-sectional view (FIG. 1a) and in a perspective view (FIG. 1b);

FIG. 2 shows a first embodiment of a medical system in a cross-sectional view;

FIGS. 3a-d show a second embodiment of a medical system in cross-sectional views (FIGS. 3a and 3c) and in perspective views (FIGS. 3b and 3d), with a removable stopper (FIGS. 3a and 3b) and with a fluidic adapter (FIGS. 3c and 3d);

FIG. 4 shows an enlarged view of a sterility cap of the medical system of FIGS. 3a and 3b in a cross-sectional view;

FIG. 5 shows an enlarged view of the sterility cap of the medical system of FIGS. 3c and 3d in a cross-sectional view;

FIG. 6 shows a flow chart of an embodiment of a method of sterility testing of a medical system;

FIG. 7 shows a flow chart of an embodiment of a method of providing a medical system; and

FIGS. 8a-b show a cross-sectional view (FIG. 8a) and a perspective view (FIG. 8b) of a subgroup forming a part of the preassembled functional module of FIGS. 1a and 1b, ready for sterilization and before assembly with the remaining parts of the functional module.

### DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

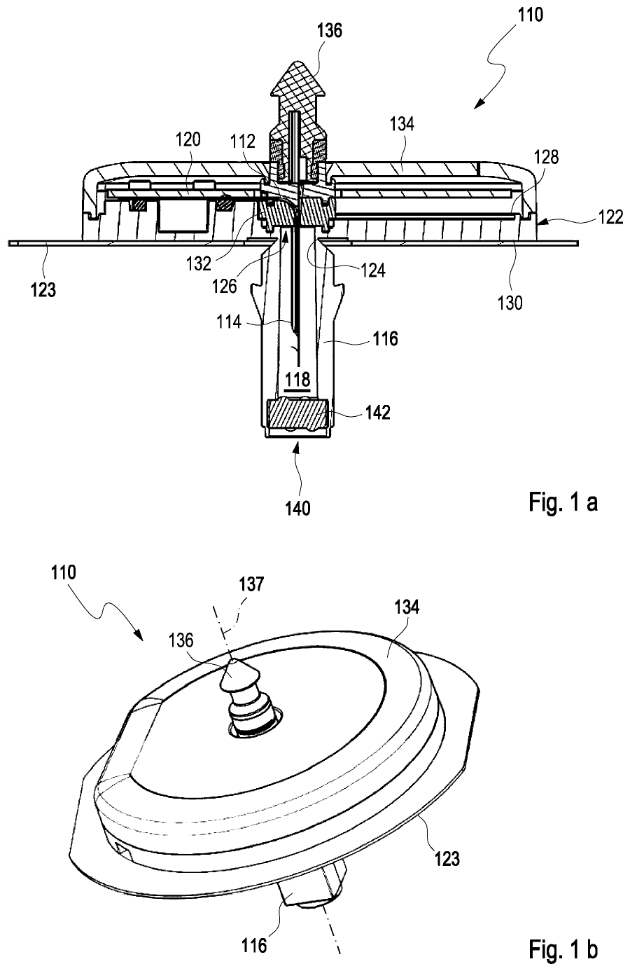

In FIGS. 1a-b, an exemplary embodiment of a preassembled functional module 110 is shown in a cross-sectional view (FIG. 1a) and in a perspective view (FIG. 1b). The preassembled functional module 110 comprises at least one analyte sensor 112 for detecting at least one analyte in a body fluid of a user. The analyte sensor 112 may be or may comprise an electrochemical sensor and may be configured for continuous monitoring of the analyte in the body fluid of the user. The analyte sensor 112 may be inserted into a body tissue of the user by using an insertion component 114 of the preassembled functional module 110, for example, an insertion cannula or an insertion needle.

The insertion component 114 is at least partially surrounded by at least one sterility cap 116. The sterility cap 116 surrounds the insertion component 114 and at least a part of the analyte sensor 112, such as a distal part or implantable part thereof. The sterility cap 116 may provide an interior space 118 which specifically may be sterilized and sealed from the outside. The insertion component 114 and at least part of the analyte sensor 112 may be located within the interior space 118.

The analyte sensor 112 is electrically connected to an electronics unit 120 of the preassembled functional module 110. The electronics unit 120 may be configured for performing at least one electronic function, specifically for performing an analyte measurement, such as by measuring at least one of an electrical current and/or a voltage provided by the analyte sensor 112. Thus, the electronics unit 120 may be configured for receiving an electronic signal, such as an electrical current and/or a voltage, from the analyte sensor 112 and further, optionally, for determining the analyte concentration in the body fluid of the user by using the electrical current and/or the voltage. The preassembled functional module 110 may further comprise at least one electrical energy storage device, for example, a battery, which specifically may be configured for supplying electrical energy to the electronics unit 120.

Further, the preassembled functional module 110 may comprise at least one base plate 122. The base plate 122, as an example, may be designed as a body mount and may comprise an adhesive surface, such as an adhesive plaster 123, for mounting the base plate 122 to a skin site of the user.

The sterility cap 116 may be connected to the base plate 122. For example, the base plate 122 and the sterility cap 116 may be manufactured as one single piece, such as by a common molding process. Alternatively, the sterility cap 116 may be connected to the base plate 122 by means of at least one of: a welding technique; a bonding technique; a soldering technique; an adhesive method. The connection between the sterility cap 116 and the base plate 122 may form a predetermined breaking point 124, such as a circumferential breaking line, as will be outlined in further detail below.

The base plate 122 may be configured for receiving the electronics unit 120 and at least part of the analyte sensor 112, such as a part configured for being inserted into the body tissue of the user. The analyte sensor 112 and the insertion component 114 may penetrate the base plate 122 via at least one through hole 126 from an upper side 128 to a lower side 130 of the base plate 122. On the lower side 130, the sterility cap 116 may surround the through hole 126. On the upper side 128, the analyte sensor 112 may be mounted to the base plate 122, such as by at least one sensor fixation element 132, whereby the sensor fixation element 132 may seal the through hole 126.

The preassembled functional module 110 may further comprise at least one cover element or cover 134. The cover 134 may cover the electronics unit 120 and at least part of the analyte sensor 112. The cover 134 may be disposed on the base plate 122 on the upper side 128.

Further, the preassembled functional module 110 may comprise at least one plunger 136 which may be part of the insertion component 114 and/or which may be connected to the insertion component 114. The plunger 136 may be movable with respect to the base plate 122 and/or with respect to a housing 146 of the medical system 144 which will be described in further detail below. The base plate 122 may be applied to the skin site of the user when the analyte sensor 112 is inserted into the body tissue of the user, such as by the adhesive plaster 123. Thus, the base plate 122 may comprise the adhesive plaster 123 for fixing the preassembled functional module 110 to the skin site of the user.

The preassembled functional module 110 may have a rotational symmetry, for example, an axial rotational symmetry about an axis 137 such as a cylinder axis, as can be seen in FIG. 1b showing the preassembled functional module 110 in a perspective view. Even though the symmetry may simplify assembly, other geometries are possible as well.

In another embodiment, the preassembled functional module 110 does not have a rotational symmetry. This may allow easier manufacturing of the medical system 144 as the exact assembling of the preassembled functional module 110 with the housing 146 is easier.

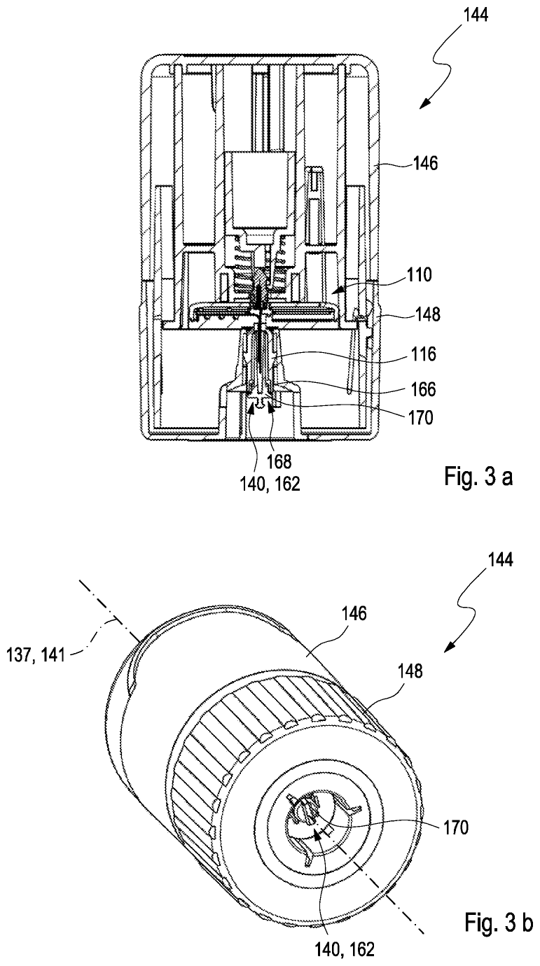

The sterility cap 116 of the preassembled functional module 110 further comprises at least one sterility testing access 140, as will be described in further detail below. The sterility testing access 140 comprises at least one of a septum 142 and a multiple-step sealing 162. In the embodiment shown in FIGS. 1a-b, the sterility testing access 140 comprises the at least one septum 142. In other embodiments, such as shown in FIGS. 3a-d, the sterility testing access 140 may comprise the at least one multiple-step sealing 162, as will be explained in further detail below.

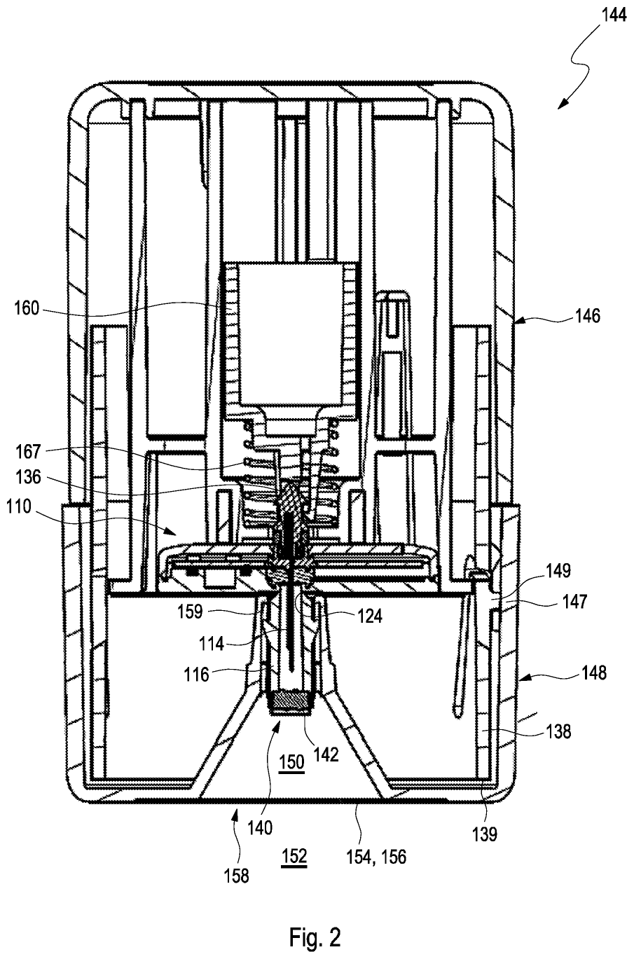

In FIG. 2, a first exemplary embodiment of a medical system 144 is shown in a cross-sectional view. The medical system 144 comprises a housing 146 which, for example, may be made of a plastic material and/or a metallic material. The housing 146 may be configured for providing protection for the enclosed parts, such as against mechanical and/or environmental influences, e.g., humidity.

The medical system 144 comprises the preassembled functional module 110, wherein the preassembled functional module 110 is received in the housing 146. The medical system 144 may comprise the embodiment of the preassembled functional module 110 shown in FIGS. 1a-b. For the description of the preassembled functional module 110, reference is therefore made to the description of FIGS. 1a-b.

The medical system 144 further comprises at least one removable protective cap 148. The removable protective cap 148 may fully or partially be made of a plastic material and/or a metallic material. The removable protective cap 148 may be connected to the housing 146 by a form-fit or a force-fit connection.

For example, a rim 147 of the removable protective cap 148 may be pushed over a rim 149 of the housing 146 or vice a versa. As another example, the removable protective cap 148 may be connected to the housing 146 via a threaded joint. The rims 147, 149 may form a tight connection or sealing. The removable protective cap 148 may be removed by the user by at least one of: pulling the removable protective cap 148 off the housing 146; turning the removable protective cap 148 off the housing 146.

Further, the removable protective cap 148 may comprise a funnel-shaped depression 150. Thus, the sterility cap 116, specifically the sterility testing access 140 of the sterility cap 116, may be accessible from an outer side 152 of the medical system 144. The sterility testing access 140 may specifically be accessible through the removable protective cap 148, more specifically through the removable protective cap 148 when the removable protective cap 148 is connected to the housing 146. In FIG. 2, the sterility testing access 140 exemplarily comprises the septum 142.

An access to the sterility cap 116 may be sealed by a removable seal 154, for example, by a removable liner 156. The removable liner 156 specifically may cover an opening 158 in the removable protective cap 148 through which the sterility cap 116 may be accessible after removing the removable seal 154. The opening 158 specifically may be an opening of the funnel-shaped depression 150.

Further, as can be seen in FIG. 2, the removable protective cap 148 may surround the sterility cap 116 within the opening 158. The opening 158 may be located within a distal surface of the removable protective cap 148 facing the skin of the user. The removable protective cap 148 may further be mechanically interlocked with the sterility cap 116 within the opening 158.

Further, the sterility cap 116 may be engaged with the removable protective cap 148, such that, when the removable protective cap 148 may be removed from the housing 146, the sterility cap 116 may be removed from preassembled functional module 110. For example, the sterility cap 116 may be mechanically interlocked with the removable protective cap 148. Thus, the mechanical interlock between the sterility cap 116 and the removable protective cap 148 may be accomplished by corresponding interlocking elements 159 of the sterility cap 116 and the removable protective cap 148, respectively, such as by one or more notches, hooks, protrusions, dents or the like, as the skilled person will recognize.

In order to prepare the medical system 144 for use, the user may remove the removable protective cap 148 from the housing 146, thereby removing the sterility cap 116 from the insertion component 114. The insertion component 114 may, thus, be revealed and the medical system 144 may be ready for use. Thereafter, the user may place a base part 138 of the housing 146 onto the skin site and actuate the insertion, as will be outlined in further detail below. As an example, the base part 138 may be or may comprise a distal edge or front rim 139, such as a circular front rim, for placement onto the skin site during insertion.

The sterility cap 116 may be arranged such that the sterility cap 116 may have at least one side in common with the removable protective cap 148. For example, the sterility testing access 140 may be located at the side which is in common with the removable protective cap 148. The side in common with the removable protective cap 148 specifically may be a distal side, facing the skin of the user.

The medical system 144 may further comprise at least one driving actuator 160. The driving actuator 160 may be configured for driving the insertion component 114 into a body tissue of the user. For example, the driving actuator 160 may engage with the plunger 136. The driving actuator 160 may be movable with respect to the base part 138 of the preassembled functional module 110. The driving actuator 160 may be movable in a forward direction, specifically in a direction towards the skin of a user. Thus, the driving actuator 160 may be configured for driving the insertion component 114 into the body tissue of the user. Further, the driving actuator 160 may be movable in a backward direction, specifically in a direction opposite to the forward direction, specifically after insertion. Thus, the driving actuator 160 may retract the insertion component 114 after the analyte sensor 112 has been inserted into the body tissue of the user, whereas the analyte sensor 112, at least with its inserted part, remains within the body tissue. The movement of the driving actuator 160, as an example, may be driven manually, such as by the user exerting a force onto the driving actuator 160. The movement into the backward direction specifically may be driven by a return spring 167.

In FIGS. 3a-d, a second exemplary embodiment of the medical system 144 is shown various views and states. The medical system 144 may, apart from the design of the sterility testing access 140, be widely identical to the first embodiment shown in FIG. 2 above, so reference may be made to the description of FIG. 2 above. FIGS. 3a and 3c show cross-sectional views of the second embodiment of the medical system 144, and FIGS. 3b and 3d show perspective views. Further, FIGS. 3a and 3b show the medical system 144 in a state in which a removable stopper 170 is attached to the sterility cap 116, which may be the normal state in which the medical system 144 may be stored, sold or used for insertion. FIGS. 3c and 3d show a second state of the medical system 144 in which the removable stopper 170 is removed and replaced by a fluidic adapter 172, for the purpose of sterility testing.

As outlined above, the medical system 144 in FIGS. 3a-d may widely correspond to the medical system 144 shown in FIG. 2, apart from the design of the sterility testing access 140. Thus, the sterility testing access 140 in the second embodiment shown in FIGS. 3a-d comprises a multiple-step sealing 162 which will be explained in further detail below.

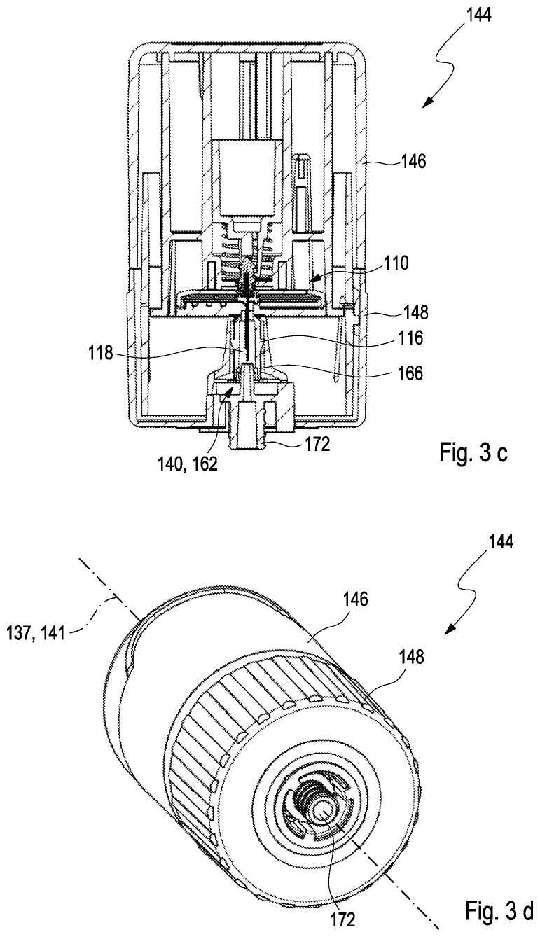

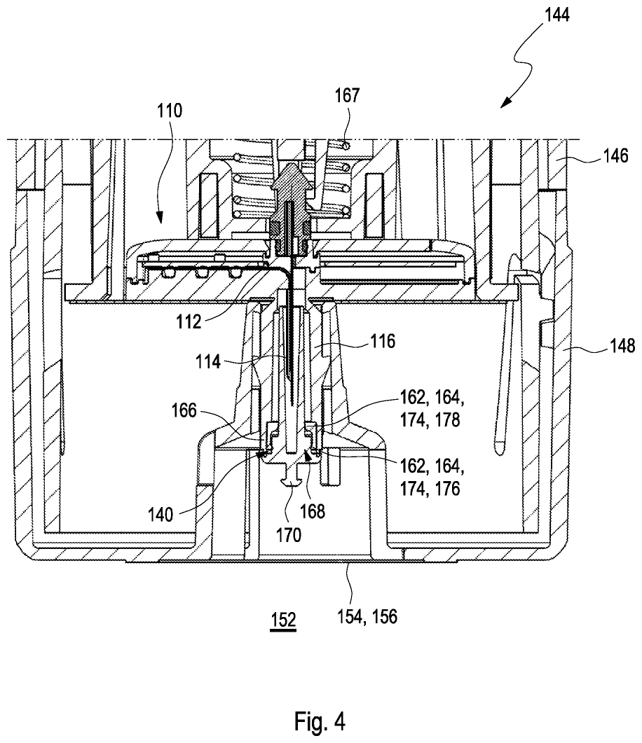

Thus, in FIG. 4, an enlarged partial view of the medical system 144 of FIGS. 3a and 3b is shown in a cross-sectional view. In FIG. 5, an enlarged view of the medical system 144 of FIGS. 3c and 3d is shown in a cross-sectional view. In the following, the multiple-step sealing 162 will be explained with respect to these FIGS. 3a-d, 4 and 5 in conjunction.

The multiple-step sealing 162, as visible, e.g., in FIGS. 4 and 5, may comprise a plurality of circumferential sealing elements 164, such as two circumferential sealing elements 164. The circumferential sealing elements 164 may be disposed concentrically, specifically with respect to an axis of extension 141 of the sterility cap 116. The axis of extension 141 of the sterility cap 116 may be identical to the axis of rotational symmetry 137, such as the cylindrical axis. The cylindrical symmetry of the medical system 144 is shown in FIG. 3b, wherein the medical system 144 is shown in a perspective view.

Further, the sterility cap 116 may comprise a tubular sidewall 166. The tubular sidewall 166 may surround a sterility testing access opening 168 located at a distal end of the sterility cap 116. The sterility testing access opening 168 may be closed by the removable stopper 170. In FIGS. 3a-b and 4, the removable stopper 170 may seal the sterility testing access opening 168. The removable stopper 170 may be removed prior to sterility testing of the medical system 144. Thus, for sterility testing, the removable stopper 170 may be replaced by a fluidic adapter 172 which may be connected to the multiple-step sealing 162. This situation is shown in FIGS. 3c-d and 5. The fluidic adapter 172 may be attached to the sterility testing access 140. The fluidic adapter 172 may be configured for inserting and removing a rinsing liquid to the interior space 118 of the sterility cap 116, such as for flushing the interior space 118 with the rinsing liquid. A detailed view of the multiple-step sealing 162 is shown in FIGS. 4 and 5.

The multiple-step sealing 162 may be configured for sealing the sterility testing access 140 of the medical system 144. As outlined above, the multiple-step sealing 162 may comprise a plurality of circumferential sealing elements 164, specifically disposed within the sterility testing access opening 168. For example, the multiple-step sealing 162 may be a two-step sealing 174. The two-step sealing 174 may comprise a first-step sealing 176 and a second-step sealing 178. The two-step sealing 174 may also comprise circumferential sealing elements 164 which may be disposed concentrically, specifically with respect to an axis of extension 141 of the sterility cap 116.

Further, the two circumferential sealing elements 164 may comprise the tubular sidewall 166 surrounding the sterility testing access opening 168. The sterility testing access 140 of the sterility cap 116 may be disposed within the sterility testing access opening 168. The sterility testing access opening 168 may be closed by the removable stopper 170. Thus, the two-step sealing 174 may be configured for receiving the removable stopper 170 at a first-step sealing 176 of the multiple-step sealing 162. The removable stopper 170 may seal the sterility testing access 140 specifically at the first-step sealing 176.

In case the sterility of the medical system 144 shall be tested, the sterility testing access 140 may be accessible from the outer side 152 of the medical system 144 through the removable protective cap 148. For this purpose, the removable protective cap 148 may remain connected to the housing 146 of the medical system 144 during sterility testing. A removal of the removable seal 154 comprised by the removable protective cap 148 may reveal the sterility testing access 140. Thus, the removable stopper 170 sealing the sterility testing access 140 may be removed from the outer side 152 of the medical system 144.

In FIG. 5, an exemplary embodiment of the fluidic adapter 172 attached to the multiple-step sealing 162 of the medical system 144 is shown. Therein, the removable stopper 170 is removed from the sterility testing access 140. The stopper 170 may be removed prior to sterility testing. After the removal of the removable stopper 170, the fluidic adapter 172 may be attached to the sterility testing access 140. The fluidic adapter 172 may be configured for inserting the rinsing liquid into the interior space 118 of the sterility cap 116. The fluidic adapter 172 may be in contact with the second-step sealing 178 of the multiple-step sealing 162. Specifically, the second-step sealing 178 may have a smaller equivalent diameter than the first-step sealing 176. Thus, the fluidic adapter 172 may not contact the first-step sealing 176. The risk of a carryover of a contamination present on the first-step sealing 176 into the interior space 118 may be minimized.

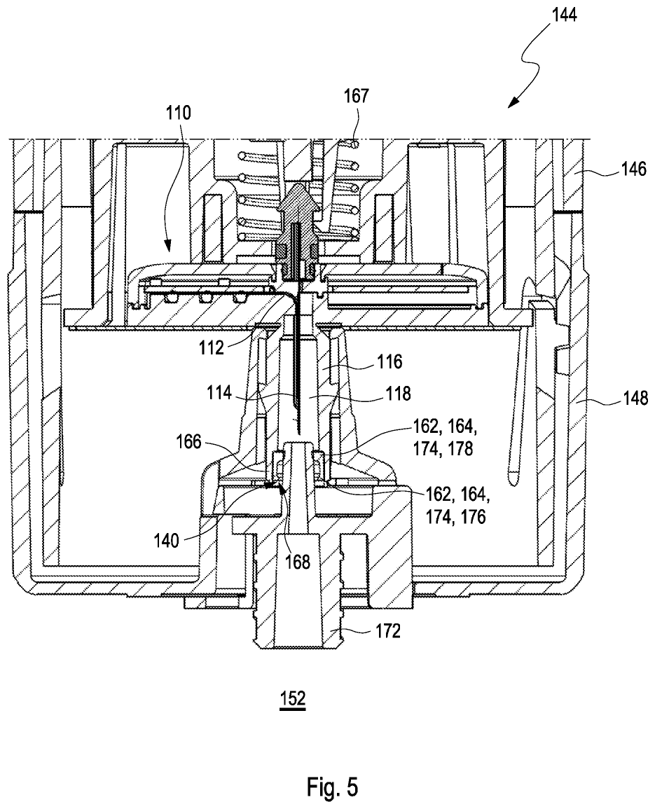

In FIG. 6, an exemplary embodiment of a method of sterility testing of the medical system 144 is shown in a flowchart. The method of sterility testing comprises the following steps, which may specifically be performed in the given order. Still, a different order may also be possible. It may be possible to perform two or more of the method steps fully or partially simultaneously. It may further be possible to perform one, more than one or even all of the method steps once or repeatedly. The method of sterility testing may comprise additional method steps that are not listed.

The medical system 144 specifically may be embodied according to any one of the embodiments disclosed above. At least, however, the medical system 144 comprises the preassembled functional module 110, wherein the preassembled functional module 110 comprises an analyte sensor 112 for detecting at least one analyte in a body fluid of a user. Further, the preassembled functional module 110 comprises at least one electronics unit 120 electrically connected to the analyte sensor 112 and an insertion component 114 for inserting the analyte sensor 112 into a body tissue of the user. The preassembled functional module 110 further comprises at least one sterility cap 116 at least partially surrounding the insertion component 114 and optionally at least a part of the analyte sensor 112.

The method of sterility testing 180 of the medical system 144 comprise the following steps:
(denoted with reference number 182) inserting the rinsing liquid into an interior space 118 of the sterility cap 116 (step A);
optionally: (denoted with reference number 184) removing the rinsing liquid from the interior space 118 of the sterility cap 116; and
(denoted with reference number 186) microbial testing of the rinsing liquid (step B).

As outlined above, step 184 is an optional step. Thus, the microbial testing in step 186 may also be performed while at least part of the rinsing liquid still is in the interior space 118, e.g., while a reservoir of the rinsing liquid is still attached to the sterility cap 116.

In step A), the rinsing liquid may be applied to the interior space 118 through the sterility testing access 140 of the sterility cap 116. Thus, step A) may further comprise attaching the fluidic adapter 172 to the sterility testing access 140. For example, the fluidic adapter 172 may be a sterile cannula which is configured for piercing the septum 142 comprised by the sterility testing access 140. The rinsing liquid may be inserted into and removed from the interior space 118 through the cannula. As another example, the fluidic adapter 172 may be embodied by the fluidic adapter 172 shown in FIG. 5. Thus, the fluidic adapter 172 may be connected to the multiple-step sealing 162 of the sterility testing access 140. The fluidic adapter 172 may provide a fluidic connection from the outer side 152 of the medical system 144 to the interior space 118 of the sterility cap 116.

Alternatively, however, the embodiment of the medical system 144 of FIG. 2 may be used. In this embodiment, step A) may be performed by inserting and removing the rinsing liquid trough the septum 142. As an example, the septum 142 may be pierced by a needle or cannula of a syringe by which the rinsing liquid is inserted into the interior space 118 and by which the rinsing liquid may also be removed from the interior space 118.

The rinsing liquid may specifically be applied to the interior space 118 without contacting the housing 146 of the medical system 144. The rinsing liquid may further be inserted to the interior space 118 in such a way that a possible contamination of the sterile parts, such as the insertion component 114 and part of the analyte sensor 112, may be incorporated into the rinsing liquid.

Step B) of the method of sterility testing may comprise incubating the rinsing liquid. Thus, a possible contamination incorporated into the rinsing liquid may be determined. After incubating the rinsing liquid, the sterility testing 180 may comprise the result, whether the insertion component 114 and the analyte sensor 112 were sterile or not.

As outlined above, step A) of the method of sterility testing may comprise attaching the fluidic adapter 172 to the sterility testing access 140 and inserting at least a part of the rinsing liquid into the interior space 118 of the sterility cap 116. In particular, at least a part of the rinsing liquid may remain outside the interior space 118 of the sterility cap 116, e.g., in a reservoir, but may fluidically be in contact with the rinsing liquid inside the interior space 118. In this state, step B) of the method may be performed. Thus, the rinsing liquid may be incubated with the fluidic adapter 172 attached to the sterility testing access 140, wherein the rinsing liquid outside the interior space 118 may be in fluidic contact with the sterile parts inside the interior space 118 of the sterility cap 116.

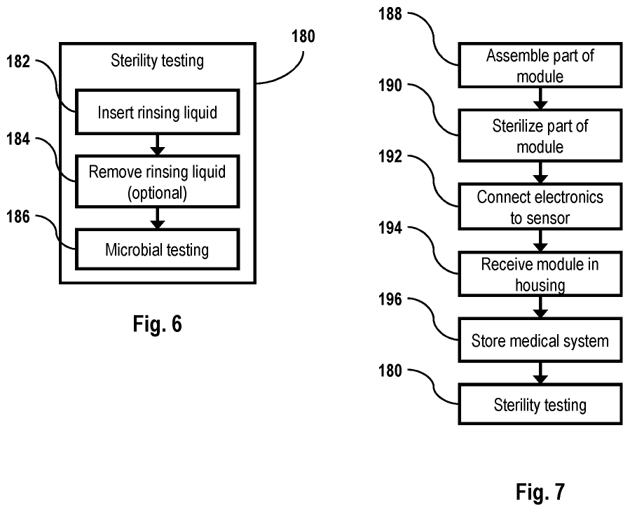

FIG. 7 shows a flowchart of an exemplary embodiment of a method of providing the medical system 144. The medical system 144 specifically may be embodied according to any one of the embodiments disclosed above. Thus, for optional details, reference may be made to the description of these embodiments. The method of providing the medical system 144 comprises the following steps, which may specifically be performed in the given order. Still, a different order may also be possible. It may be possible to perform two or more of the method steps fully or partially simultaneously. It may further be possible to perform one, more than one or even all of the method steps once or repeatedly. The method of providing the medical system 144 may comprise additional method steps that are not listed.

The method of providing the medical system 144 comprises the following steps:
I. (denoted with reference number 188) assembling a part 195 of the preassembled functional module 110, the part 195 of the preassembled functional module 110 comprising the analyte sensor 112 and the insertion component 114 as well as the sterility cap 116 at least partially surrounding the insertion component 114;
II. (denoted with reference number 190) sterilizing the part 195 of the preassembled functional module 110, specifically by using one or more of e-beam or gamma sterilization;
III. (denoted with reference number 192) assembling the preassembled functional module 110, the assembling comprising electrically connecting the electronics unit 120 to the analyte sensor 112; and
IV. (denoted with reference number 194) assembling the medical system 144, the assembling comprising receiving the preassembled functional module 110 in the housing 146 and connecting the removable protective cap 148 to the housing 146.

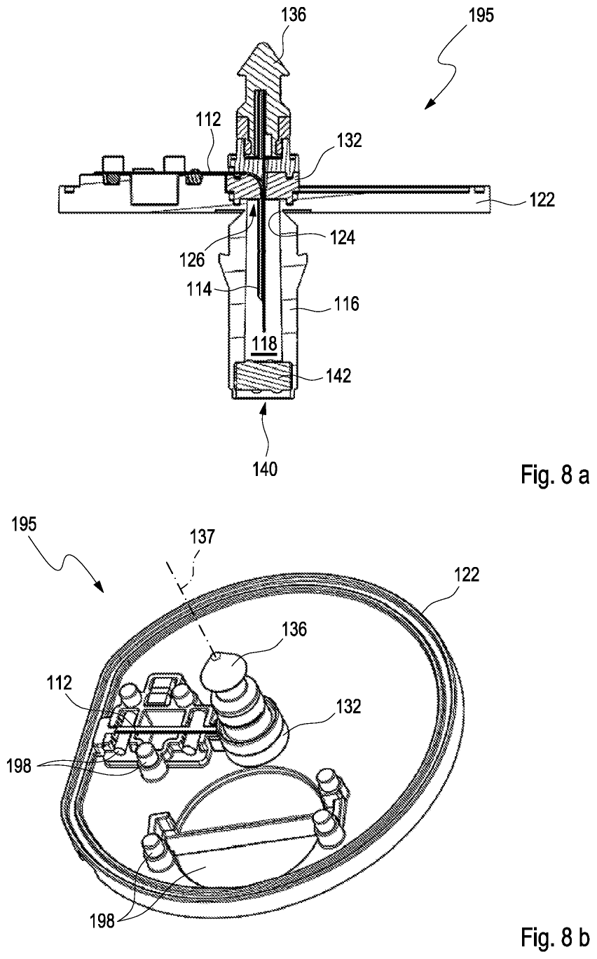

Step I. of the method of providing the medical system 144 may refer to an assembly of a part of the medical system 144 which comprises at least some of the components to be sterilized. The part 195 assembled in step I. may form a subgroup of the preassembled functional module 110. In FIGS. 8a and 8b, an exemplary embodiment of this part 195 or subgroup of the preassembled functional module 110 is shown in a cross-sectional view (FIG. 8a) and in a perspective view (FIG. 8b). Therein, the preassembled functional module 110 corresponds to the embodiment of FIGS. 1a-b and 2. As the skilled person will recognize, however, the subgroup may also be embodied, within the scope of this disclosure, in other ways, such as according to the embodiment shown in FIGS. 3a-d, 4 and 5.

The part 195 or subgroup, as shown in FIGS. 8a-b, comprises at least some of the sterile parts of the medical system 144. The sterile parts may comprise at least the analyte sensor 112, the insertion component 114 and/or the sterility cap 116. Further, the subgroup may comprise the sensor fixation element 132 and/or the base plate 122. As can be seen in FIG. 8b, the base plate 122, as an example, may comprise one or more structural elements 198, such as for mounting the electronic unit 120, for electrically contacting the analyte sensor 112, for receiving an electrical energy storage device or the like. The subgroup may be assembled under clean conditions, such as in a clean room facility or the like.

After performing step I., i.e., after assembling part 195, part 195 may be subjected to the sterilization in step II. For this purpose, as an example, the subgroup may be transferred to a sterilization unit, such as an X-ray sterilizer, a gamma sterilizer, an e-beam sterilizer or the like. At this stage, preferably, no components which are prone to electronic damages by the sterilization process are present in the subgroup, such as the electronics unit 120.

After performing step II., the part 195, also referred to as the subgroup, is a sterilized part or sterilized subgroup. Thus, specifically, the interior space 118 is in a sterilized condition, including the parts received therein, such as the insertion component 114 and the part of the analyte sensor 112 received therein.

The sterilized part 195 may then undergo further processing, wherein, however, the sterilized state of at least the interior space 118 and the parts received therein remains unchanged.

Thus, in step III., the electronics unit 120 may be attached to the part 195. Further, the cover 134 may be mounted, thereby closing the preassembled functional module 110. Further, the adhesive plaster 123 may be applied to the base plate 122.

In step IV., the preassembled functional module 110 may be combined with other components of the medical system 144. Thus, the preassembled functional module 110 may be inserted into the housing 146. Further, the removable protective cap 148 may be attached to the housing 146, and the removable seal 154 may be applied.

Thus, the method of providing the medical system 144 may comprise an assembly process wherein sterile and non-sterile part may be processed.

The method of providing the medical system 144 may further comprise:
V. (denoted by reference number 196) storing the medical system 144.

After storing the medical system 144, the method of providing the medical system 144 may further comprise:
VI. (denoted by reference number 180) sterility testing of the medical system 144 by using the method of sterility testing of a medical system 144.

The sterility of the medical system 144 may be tested by performing step VI. Step VI. may be performed on few selected medical systems 144. The medical systems 144 subjected to sterility testing 180 may be selected on a random basis, specifically in such a way that the sterility testing 180 of the few selected medical systems 144 may allow for verifying the sterility of all assembled medical systems 144. For example, a percentage of sterility tested medical systems 144 to assembled medical systems 144 may be less than 1%, e.g., 0.1% or even less.

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

### LIST OF REFERENCE NUMBERS

110 preassembled functional module
112 analyte sensor
114 insertion component
116 sterility cap
118 interior space
120 electronics unit
122 base plate
123 adhesive plaster
124 predetermined breaking point
126 through hole
128 upper side
130 lower side
132 sensor fixation element
134 cover
136 plunger
137 symmetry axis
138 base part
139 front rim
140 sterility testing access
141 axis of extension
142 septum
144 medical system
146 housing
147 rim of the removable protective cap
148 removable protective cap
149 rim of the housing
150 funnel-shaped depression
152 outer side
154 removable seal
156 removable liner
158 opening
159 interlocking element
160 driving actuator
162 multiple-step sealing
164 circumferential sealing elements
166 tubular sidewall
167 return spring
168 sterility testing access opening
170 removable stopper
172 fluidic adapter
174 two-step sealing
176 first-step sealing
178 second-step sealing
180 sterility testing
182 inserting a rinsing liquid
184 removing a rinsing liquid
186 microbial testing of a rinsing liquid
188 assembling a part of a preassembled functional module
190 sterilizing a part of a preassembled functional module
192 assembling a preassembled functional module
194 assembling a medical system
195 part of the preassembled functional module
196 storing a medical system
198 structural elements

What is claimed is:

1. A medical system, comprising:
a housing;
a module received in the housing and comprising:
an analyte sensor configured for detecting an analyte in a body fluid of a user,
an electronics unit electrically connected to the analyte sensor,
an insertion component configured for inserting the analyte sensor into body tissue of the user, and
a sterility cap at least partially surrounding the insertion component;
a removable protective cap connected to the housing and covering the module, wherein the protective cap at least partially surrounds the sterility cap; and
wherein the sterility cap comprises a sterility testing access that has at least one of a septum and a multiple-step sealing.

2. The medical system according to claim 1, wherein the sterility cap comprises the multiple-step sealing and the multiple-step sealing further comprises a plurality of circumferential sealing elements having differing equivalent diameters, the circumferential sealing elements being disposed within an interior space of the sterility cap.

3. The medical system according to claim 1, wherein the sterility cap comprises a tubular sidewall surrounding a sterility testing access opening that is closed by at least one of the septum and a removable stopper.

4. The medical system according to claim 1, wherein the sterility cap is accessible from an outer side of the medical system and wherein the sterility cap is sealed by a removable seal.

5. The medical system according to claim 1, wherein the sterility cap is engaged with the removable protective cap such that when the protective cap is removed from the housing, the sterility cap is removed from the module.

6. The medical system according to claim 1, wherein the sterility cap has at least one side in common with the protective cap.

7. The medical system according to claim 1, wherein the module further comprises a base plate, wherein the sterility cap is connected to the base plate.

8. The medical system according to claim 1, wherein the module further comprises a cover that covers the electronics unit and at least part of the analyte sensor.

9. The medical system according to claim 1, wherein the sterility cap provides a sealed interior space, wherein the analyte sensor and the insertion component are at least partially located within the sealed interior space.

10. The medical system according to claim 1, further comprising a driving actuator configured for driving the insertion component into the body tissue of the user.

11. The medical system according to claim 1, wherein the module further comprises an electrical energy storage device.

12. A method of sterility testing a medical system having a module that has an electronics unit electrically connected to an analyte sensor, an insertion component for inserting the analyte sensor into body tissue of the user, and a sterility cap that at least partially surrounds the insertion component, the method comprising:
A) inserting a rinsing liquid into an interior space of the sterility cap; and
B) microbial testing of the rinsing liquid.

13. The method according to claim 12, wherein the sterility cap has a sterility testing access, wherein step A) comprises attaching a fluidic adapter to the sterility testing access, wherein the sterility testing access comprises at least one multiple-step sealing, wherein step A) comprises removing at least one stopper from the sterility testing access, the stopper being in contact with a first-step sealing of the multiple-step sealing, step A) further comprising attaching the fluidic adapter to the sterility testing access, wherein the fluidic adapter is in contact with a second-step sealing of the multiple-step sealing, wherein the first-step sealing has a larger equivalent diameter than the second-step sealing.

14. A method of providing the medical system according to claim 1, the method comprising:
I. assembling a part of the module that includes the analyte sensor, the insertion component and the sterility cap at least partially surrounding the insertion component;
II. sterilizing the part of the module;
III. assembling the module, the assembling comprising electrically connecting the electronics unit to the analyte sensor; and
IV. assembling the medical system by receiving the module in the housing and connecting the removable protective cap to the housing.

15. The method according to claim 14, the method further comprising storing the medical system.
